(19) United States
(12) Patent Application Publication
(10) Pub. No.: US 2004/0241832 A1
(43) Pub. Date: Dec. 2, 2004

(54) Cell culture detection apparatus, cell culture observation apparatus, and cell culture observation method

(75) Inventors: Kayuri Muraki, Tokyo (JP); Hiroyuki Imabayashi, Tokyo (JP)

Correspondence Address: SCULLY SCOTT MURPHY & PRESSER, PC, 400 GARDEN CITY PLAZA, GARDEN CITY, NY, 11530

(73) Assignee: OLYMPUS CORPORATION, TOKYO (JP)

(21) Appl. No.: 10/856,020

(22) Filed: May 28, 2004

(30) Foreign Application Priority Data
Jun. 2, 2003 (JP) 2003-156795
Jul. 11, 2003 (JP) 2003-273674

(51) Int. Cl. C12M 1/34

(52) U.S. Cl. 435/287.1

(57) ABSTRACT

A cell culture detection apparatus includes a culture vessel that houses cells together with a culture liquid, a culturing device that cultures the cells under predetermined culturing conditions, a detection device that detects a feature of the cells among the cells being cultured, and a light blocking device that blocks the culture vessel from environmental light when the feature is not detected.

### BACKGROUND OF THE INVENTION

1. Field of the Invention

The present invention relates to a cell culture detection apparatus that detects information according to the reaction of cells during culturing. The present invention also relates to a cell culture observation apparatus and cell culture observation method for observing information obtained from reactions of cell culturing while culturing cells.

Priority is claimed on Japanese Patent Application No. 2003-156795, filed Jun. 2, 2003, and Japanese Patent Application No. 2003-273674, filed Jul. 11, 2003, the contents of which is incorporated herein by reference.

2. Description of Related Art

Accompanying the progress made in the field of gene analysis technology in recent years, together with determining the gene sequences of numerous living organisms including humans, the cause-and-effect relationships between proteins and other gene products and diseases have begun to be elucidated. In addition, in order to more comprehensively and statistically analyze various proteins and genes in the future, it will be necessary to detect predetermined information while culturing cells for extended periods of time. Consequently, there is a need for an apparatus that allows cells to be cultured and observed microscopically.

A known example of this type of apparatus involves the use of a transparent, constant-temperature culture vessel for microscopic observation that allows the setting of culturing conditions for various types of cells (see, for example, Japanese Unexamined Patent Application, First Publication No. 10-28576).

This transparent, constant-temperature culture vessel for microscopic observation has a pair of transparent heating plates that can be controlled to a predetermined temperature by a temperature controller, a carbon dioxide supply port and discharge port for adjusting the concentration of carbon dioxide within the vessel, and an evaporation dish for maintaining the humidity in the vessel that is sealed with a sealing gasket.

When observing cells using this transparent, constant-temperature culture vessel for microscopic observation, since the temperature, carbon dioxide concentration and humidity within the vessel can be controlled, cells can be observed while they are being cultured. Namely, by observing the cells with an objective lens from below the transparent heating plate, for example, the time-based changes in cell culturing status can be observed.

Thus, by being able to observe cells using the aforementioned transparent constant-temperature culture vessel for microscopic observation, when observing or recording photographic records of the culturing status of various cells in the research fields of biology, reproduction or bacteriology and so forth, observation and recording of time-based changes can be carried out both continuously and easily by controlling temperature, carbon dioxide concentration and humidity as desired and making various settings for the culturing status while observing the cells microscopically.

In particular, different from genes and so forth, since cells allow the detection of fluorescence in the viable state, such as detection of the expression of green fluorescent protein (GFP) within cells, to be frequently used as a measuring method, management of environmental conditions for culturing cells is an important factor for obtaining accurate measurement results. Thus, management of temperature and carbon dioxide concentration as previously mentioned is essential for culture vessels such as plastic or glass Petri dishes and Petri plates arranged under a microscope so as not to cause the cells to be destroyed by microscopic observation over a long period of time.

In addition, cells have various properties according to their type, and there are cells that have properties that are extremely susceptible to changes in the external environment, for example. There are cases in which these cells may be easily destroyed by, for example, heating procedures or uneven temperature distribution caused by a sudden rise in temperature. Although varying according to the type of cells, it is typically necessary to maintain the cells at a constant temperature of 37±0.5° C. and a constant carbon dioxide concentration of 3-8% as conditions for cell culturing. In addition, in addition to temperature and carbon dioxide concentration, other managed environmental factors can also not be ignored. For example, the effect of light such as sunlight and indoor light is also an important management parameter. In other words, phototoxicity resulting from prolonged irradiation with light causes an increase in the levels of active enzymes within the cells thereby having an effect on cell growth. In addition, during long-term cell culturing, semi-batch replacement of the culture liquid without causing contamination by dust particles and so forth is also an important management parameter for the conditions of the culturing environment.

As another method for observing cells, a method is known in which cells are inoculated into a plastic or glass dish or flask followed by culturing in an incubator. The inside of this incubator is set to, for example, a carbon dioxide concentration of 5%, temperature of 37° C. and humidity of 100%, and an environment is maintained that is suitable for cell growth. Moreover, together with imparting nutrients to the cells, the culture liquid is replaced every 2 to 3 days to maintain a pH suitable for culturing.

Although several methods are known for observing cells during culturing, one example involves removing the aforementioned dish or flask from the incubator and observing the cells using an inverted microscope such as phase contrast microscope. In this method, it is necessary to observe the cells as quickly as possible and return the cells to the incubator following completion of observation.

This is to prevent the activity of the cells from being impaired due to the cells being placed in an ordinary environment for a long period of time. Namely, it is difficult to make an accurate evaluation if cell activity becomes unstable. In addition, when removing the cells from the incubator, it is also necessary to take adequate precautions with respect to preventing contamination and so forth.

In addition, another known observation method involves evaluating cells in a dish that differs for each measurement. Namely, in the case of detecting time-based changes in cells, a large number of dishes inoculated with cells under the same conditions are prepared, and the cells are evaluated by removing each dish from an incubator at each predetermined measurement time.

In this method, together with one or several dishes of cells being used in a single observation, since cell activity may be impaired due to various manipulations made for the purpose of observation, typically only one dish is used for a single measurement.

### SUMMARY OF THE INVENTION

The present invention provides a cell culture detection apparatus including: a culture vessel that houses cells together with a culture liquid, a culturing device that cultures the cells under predetermined culturing conditions, a detection device that detects a feature of the cells among the cells being cultured, and a light blocking device that blocks the culture vessel from environmental light when the feature is not detected.

The present invention provides a cell culture detection apparatus including: a culture vessel that houses cells together with a culture liquid, a culturing device that cultures the cells under predetermined culturing conditions, and a detection device that detects a feature of the cells among the cells being cultured; wherein, the culturing device has a warming device having a temperature sensor that measures the temperature of the culture vessel, and at least one of either a culture vessel warming unit that warms the culture vessel, a line warming unit that warms a line that supplies or discharges the culture liquid within the culture vessel, or a culture liquid warming unit that warms the culture liquid; and, the warming device controls the temperature to a predetermined temperature based on the temperature measured with the temperature sensor.

The present invention provides a cell culture detection apparatus including: a culture vessel that houses cells together with a culture liquid, a culturing device that cultures the cells under predetermined culturing conditions, and a detection device that detects a feature of the cells among the cells being cultured; wherein, the culturing device has a temperature sensor that measures the temperature of the culture vessel, and a culture vessel warming unit that warms the culture vessel, and the culture vessel warming unit blows warm air towards the outer surface of the culture vessel based on the temperature measured with the temperature sensor.

The present invention provides a cell culture observation apparatus for continuously observing time-based changes of one or a plurality or cells present on a support or in a solution; including: a culture vessel that houses the cells and is capable of maintaining cell activity; a movable stage that holds the culture vessel; an imaging section that captures images of the cells in the culture vessel by dividing into each region corresponding to each cell; and, an analysis section that analyzes the cells by at least extracting a geometrical feature or an optical feature of the cells within a region based on the images of each region captured by the imaging section.

The present invention provides a cell culture observation method for continuously observing time-based changes in one or a plurality of cells present on a support or in a solution while culturing cells in a culture vessel; including: an imaging step wherein images of the cells in the culture vessel are captured by dividing into each region corresponding to each cell; and an analysis step wherein the cells are analyzed by extracting at least a geometrical feature or an optical feature of the cells in each region captured in the imaging step.

### BRIEF DESCRIPTION OF THE DRAWINGS

FIG. 1 is a block diagram showing a first embodiment of a cell culture detection apparatus according to the present invention.

FIG. 2 is a-perspective view showing the state in which a culture vessel of the cell culture detection apparatus shown in FIG. 1 is installed in a case.

FIG. 3 is a cross-sectional view showing the state in which a cell feature is being detected.

FIGS. 4A and 4B are drawings showing the relationship between a culture vessel and a light blocking device, in particular, FIG. 4A is a side view of the culture vessel and a light blocking unit, and FIG. 4B is a drawing of FIG. 4A taken along arrows B-B.

FIG. 5 is a cross-sectional view showing the state in which a culture vessel is housed within a light blocking unit.

FIG. 6 is a cross-sectional view showing the state in which auto-fluorescence of a culture liquid is being measured.

FIGS. 7A and 7B are drawings showing the relationship between a culture vessel and a light blocking device in a second embodiment of a cell culture detection apparatus according to the present invention, in particular, FIG. 7A is a side view of the culture vessel and a light blocking unit, and FIG. 7B is a drawing of FIG. 7A taken along arrows C-C.

FIG. 8 is a block diagram showing a third embodiment of a cell culture detection apparatus according to the present invention.

FIGS. 9A and 9B are drawings showing the 96-well microtiter plate shown in FIG. 8, in particular, FIG. 9A is a cross-sectional view, and FIG. 9B is a drawing of FIG. 9A taken along arrows D-D.

FIG 10 is a drawing showing a variation of the discrimination section of a cell culture detection apparatus according to the present invention.

FIG. 11 is a block diagram showing one example of a cell culture observation apparatus according to the present invention.

FIG. 12 is a schematic drawing showing the cell culture observation apparatus shown in FIG. 1.

FIG. 13 is an overhead view showing the state in which a culture vessel is fastened to a culture vessel mounting section.

FIG. 14 is a cross-sectional view showing the state in which a culture vessel is fastened to a culture vessel mounting section.

FIG. 15 is a perspective view showing a flow straightening member disposed in a culture vessel.

FIG. 16 is a drawing showing the measuring range and imaging step during observation of cells on a slide glass.

FIG. 17 is a drawing showing the relationship between measuring range and areas

FIG. 18 is a flow chart showing a cell culture observation method according to the cell culture observation apparatus shown in FIG. 11.

FIG. 19 is a flow chart used when moving a stage in the flow chart shown in FIG. 18.

FIG. 20 is a flow chart used when starting measurement in the flow chart shown in FIG. 8.

FIG. 21 is a flow chart showing processing by an image processing section in the case of observing time-based changes of cells.

FIG. 22 is a flow chart showing processing by a data processing section in the case of observing time-based changes of cells.

FIG. 23 is a drawing showing a variation of the measuring range and imaging step during observation of cells on a slide glass.

### DETAILED DESCRIPTION OF THE INVENTION

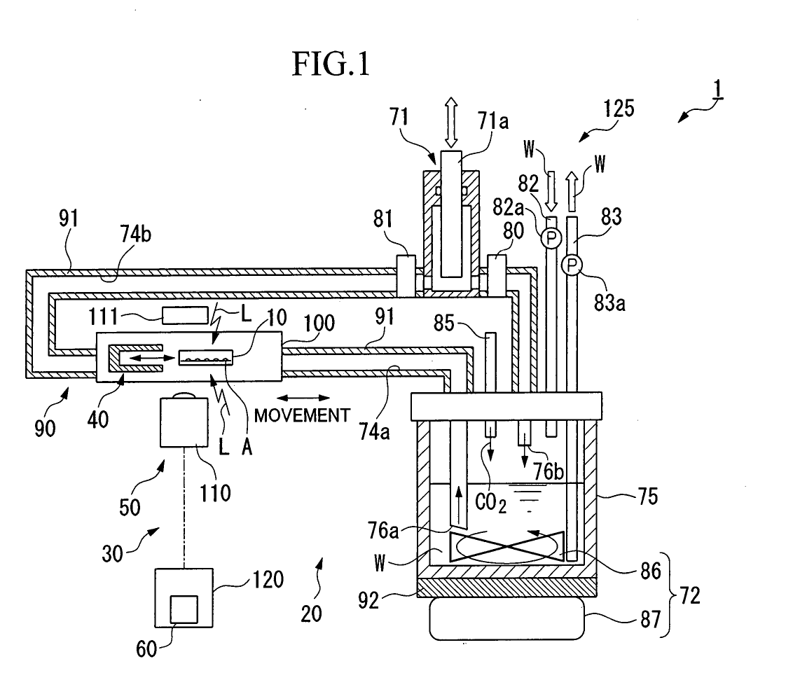

The following provides an explanation of one embodiment of the cell culture detection apparatus according to the present invention with reference to FIGS. 1 to 6.

As shown in FIG. 1, cell culture detection apparatus 1 of the present embodiment is provided with a culture vessel 10 that houses cells A together with culture liquid W, a culturing device 20 that cultures the cells A under predetermined culturing conditions, a detection device 30 that detects a feature of the cells A among cells A being cultured, a light blocking device 40 that blocks environmental light L from culture vessel 10 when the feature is not detected, a measuring device 50 that measures the level of auto-fluorescence of culture liquid W, and a discrimination section (discrimination device) 60 that judges whether or not culture liquid W has degraded based on the measurement results obtained from the measuring device 50.

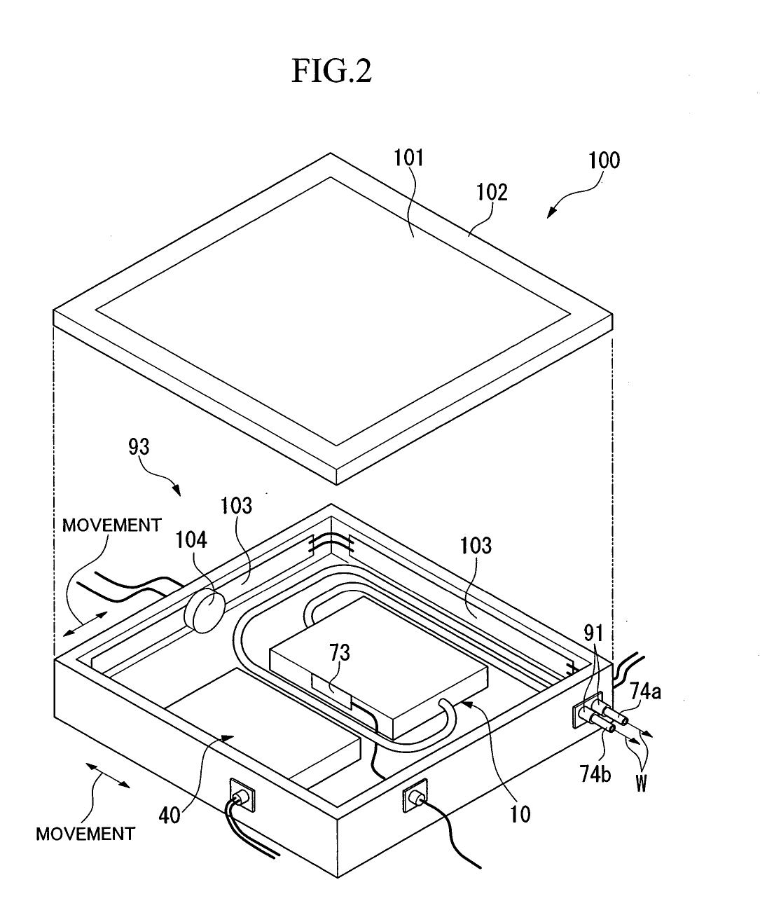

As shown in FIGS. 1 and 2, the aforementioned culture vessel 10 is housed within a case 100 formed into a rectangular shape that is sufficiently larger than the culture vessel 10. In addition, this case 100 is fastened on an X-Y stage of an inverted microscope not shown. As a result, culture vessel 10 and case 100 are able to move in the horizontal direction in synchronization with X-Y scanning of the X-Y stage. Furthermore, a more detailed of this case 100 is provided hereinafter.

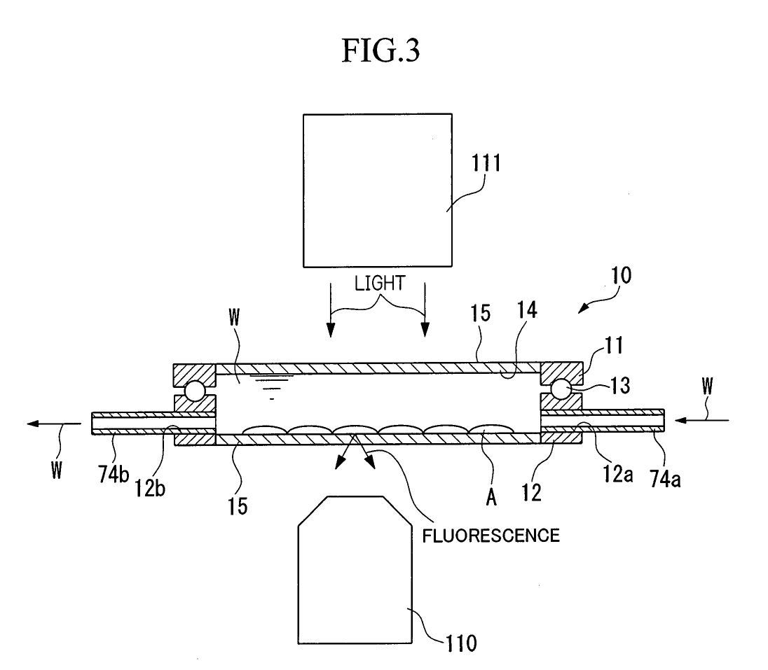

As shown in FIG. 3, culture vessel 10 is composed in the shape of a box having an internal space 14 by a culture vessel upper frame 11 and culture vessel lower frame 12, which are formed from a material not having cytotoxicity such as Teflon (registered trade name), PEEK (registered trade name) or corrosion-resistant stainless steel, being mutually connected in the vertical direction by screws or other fastening members not shown by means of an O-ring 13. In addition, glass members 15, which each have an optically flat surface, are joined to culture vessel upper frame 11 and culture vessel lower frame 12 by an adhesive and so forth not having cytotoxicity. Namely, the upper and lower surfaces of culture vessel 10 are covered by a pair of glass members 15. Furthermore, the adhesive used to adhere the pair of glass members 15 is preferably an adhesive able to withstand autoclave conditions (e.g., 120° C., 4 atm).

In addition, culture liquid support port 12a, which supplies culture liquid W to internal space 14, and culture liquid discharge port 12b, which discharges culture liquid W from internal space 14, are respectively formed on both sides of culture vessel lower frame 12.

As shown in FIGS. 1 and 2, the aforementioned culturing device 20 is provided with a syringe piston pump (circulation pump) 71 that circulates culture liquid W, a stirring unit 72 that maintains the carbon dioxide concentration in culture liquid W at a predetermined concentration by stirring the culture liquid W, a temperature sensor 73 that measures the temperature of culture vessel 10, and a warming device 90 that warms culture vessel 10. In addition, the warming device 90 has a cylindrical heater (line warming unit) 91 that warms a line for supply or discharging culture liquid 10 in culture vessel 10, a culture liquid tank heater (culture liquid warming unit) 92 that warms culture liquid W, and a culture vessel warming unit 93 that warms culture vessel 10.

Here, as shown in FIG. 3, one end of flexibly formed culture liquid supply line 74a is connected to culture liquid supply port 12a of culture vessel 10, while one end of flexibly formed culture liquid discharge line 74a is connected to culture liquid discharge port 12b. As shown in FIG. 1, the other end of the aforementioned culture liquid supply line 74a is immersed in culture liquid W held inside culture liquid tank 75. Namely, the other end of culture liquid supply line 74a serves as a supply port 76a of culture liquid W. In addition, the other end of the aforementioned culture liquid discharge line 74b is inserted into culture liquid tank 75 with the aforementioned syringe piston pump 71 interposed therein. Namely, the other end of culture liquid discharge line 74b serves as a discharge port 76b of culture liquid W.

The aforementioned syringe piston pump 71 is a non-pulsating circulation pump that pumps culture liquid W without generating pressure fluctuations, and has a vertically moving piston 71a inside. In addition, solenoid valves 80 and 81 are interposed on both sides of syringe piston pump 71 in culture liquid discharge line 74b. One solenoid valve 80 is interposed on the side of culture liquid tank 75, while the other solenoid valve 81 is interposed on the side of culture vessel 10. Piston 71a and both solenoid valves 80 and 81 are integrally controlled by a personal computer (PC) 120.

For example, when piston 71a is operated (in the upward direction relative to the paper) so that culture liquid W is filled into syringe piston pump 71 with one solenoid valve 80 closed and the other solenoid valve 81 open, culture liquid W is aspirated from supply port 76a resulting in a flow that supplies culture liquid W from culture liquid supply line 74a into internal space 14 of culture vessel 10.

In this manner, after culture liquid W is supplied into culture vessel 10 by syringe piston pump 71 and both solenoid valves 80 and 81 by flowing from supply port 76a through culture liquid supply line 74a, it flows through culture liquid discharge line 74b and again returns to culture liquid tank 75 from discharge port 76b to form a circulating system.

In addition, the flow rate of culture liquid W can be set as desired to an arbitrary flow rate such as a low rate of about 1 ml/30 min by changing the movement rate of piston 71a. Furthermore, the direction of the supply and discharge of culture liquid W can be switched by reversing the aforementioned timing.

The aforementioned culture liquid tank 75 is formed so that the inside is sealed from a material having superior thermal conductivity such as corrosion-resistant stainless steel or glass. In addition, tank supply line 82, which supplies fresh culture liquid inside, and tank discharge line 83, which discharges culture liquid W from culture liquid tank 75, are provided in culture liquid tank 75. Furthermore, tank discharge line 83 is provided so as to be located near the bottom of culture liquid tank 75. The base of the aforementioned tank supply line 82 is connected to a culture liquid supply source not shown, and enables culture liquid to be supplied from the culture liquid supply source into culture liquid tank 75 by a tank supply pump 82a. In addition, the base of tank discharge line 83 is connected to a discharge tank not shown, and enables culture liquid to be discharged from culture liquid tank 75 by a tank discharge pump 83a.

The driving of the aforementioned tank supply pump 82a and tank discharge pump 83a is controlled by PC 120, the culture liquid W inside culture liquid tank 75 can be replaced or replenished automatically by receiving a signal from PC 120.

In addition, the aforementioned cylindrical heater 91, which warms the aforementioned culture liquid supply line 74a and culture liquid discharge line 74b, is disposed around both lines 74a and 74b over nearly their entire length. This cylindrical heater 91 warms culture liquid W that flows therein by warming both lines 74a and 74b, and has the function of warming culture vessel 10 by means of the culture liquid W. The temperature of this cylindrical heater 91 is controlled by PC 120.

In addition, a carbon dioxide supply line 85, which supplies carbon dioxide at a prescribed concentration (e.g., 5%) to culture liquid tank 75, is provided in the culture liquid tank 75. This carbon dioxide supply line 85 supplies carbon dioxide from a carbon dioxide supply source not shown arranged outside. In addition, the aforementioned culture liquid tank heater 92 is provided below culture liquid tank 75. This culture liquid heater 92 has the function of warming culture liquid W inside culture liquid tank 75 having superior thermal conductivity. Furthermore, the temperature of this culture liquid tank heater 92 is controlled by PC 120.

Moreover, a stirrer 86 that is rotated by the rotation of a magnet is rotatably attached to the bottom of culture liquid tank 75, and is rotated by the magnetic field generated from magnetic stirrer 87 attached to the lower portion of culture tank heater 92. Namely, this stirrer 86 and magnetic stirrer 87 compose the aforementioned stirring unit 72.

As shown in FIG. 2, the previously described case 100 is formed from a metal such as aluminum having superior thermal conductivity, and the lower surface on which culture vessel 10 is installed is provided with an optically transparent thin glass plate or opening not shown. As a result, culture vessel 10 can be observed from the lower surface of case 100. In addition, a cover member 102 having a glass member 101 is removably attached to the upper portion of case 100. As a result, culture vessel 10 can be accessed while inside case 100.

The aforementioned temperature sensor 73 and the aforementioned light blocking device 40 are disposed within case 100. Temperature sensor 73 is of, for example, a movable type so as to make contact by being fastened to a spring member and so forth utilizing the force of the spring, and measures temperature by contacting a lateral surface of culture vessel 10 when culture vessel 10 is installed. This temperature sensor 73 has a function that transmits the measured temperature of culture vessel 10 to PC 120.

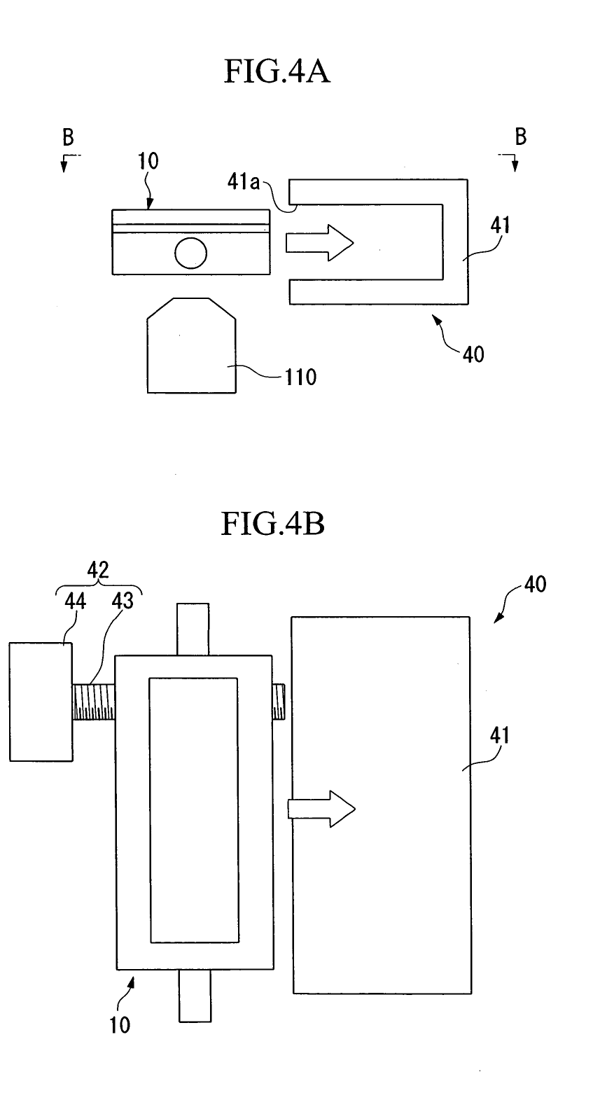

As shown in FIG. 4, the aforementioned light blocking device 40 has a light blocking unit 41 that blocks light from the periphery of culture vessel 10, and a culture vessel transport device 42 that transports culture vessel 10 to within light blocking unit 41.

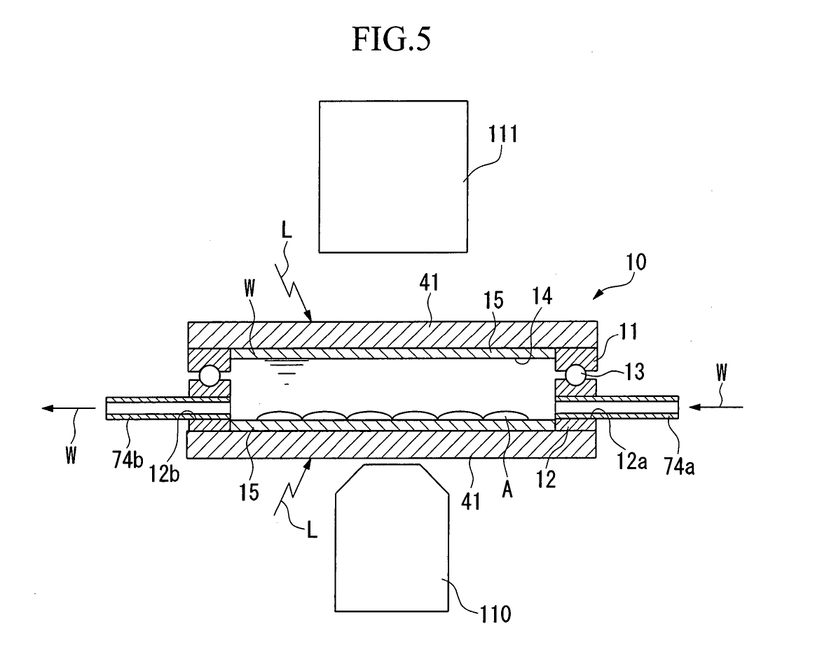

Light blocking unit 41 is formed from a material that is optically impenetrable to light in a shape having a U-shaped cross-section and of a size that enables culture vessel 10 to be housed inside, and is fixed within case 100 with opening 41a facing towards culture vessel 10. In addition, the height of opening 41 a is formed so as to be of nearly the same height as the thickness of culture vessel 10. Namely, as shown in FIG. 5, light blocking unit 41 blocks light from the entire surfaces of the pair of glass members 15 arranged on the upper and lower surfaces of culture vessel 10, and has a function that prevents indoor light or other environmental light L from entering internal space 14.

In addition, as shown in FIG. 4(b), a ball screw 43 is provided between light blocking unit 41 and culture vessel 10, and the ball screw 43 is rotatably locked in a locking portion not shown of culture vessel 10. One end of the ball screw 43 is linked to motor 44. In other words, as a result of turning ball screw 43 by driving motor 44, culture vessel 10 can be housed within light blocking unit 41 by moving from an observation position within case 100 to within light blocking unit 41. Namely, this ball screw 43 and motor 44 compose the aforementioned culture vessel transport device 42. In addition, the driving of motor 44 is controlled by PC 120.

In addition, as shown in FIG. 2, a case warming heater 103 that warms case 100 itself as well as the internal space of case 100 is disposed around the entire periphery of the inside surface of case 100. The temperature of this case warming heater 103 is controlled by PC 120. Moreover, a fan 104 that agitates the air inside case 100 is provided inside case 100. Namely, fan 104 has a function that blows warm air inside case 100 that has been warmed by case warming heater 103 towards the outer surface of culture vessel 10. Furthermore, the operation of fan 104 is controlled by PC 120 to control the amount of warm air blown.

Furthermore, an output cable of case warming heater 103, a power cable of fan 104, and connecting sections such as connectors not shown that connect culture liquid supply line 74a and culture liquid discharge line 74b are provided outside of case 100 to reliably and easily connect the outside and inside. In addition, culture liquid supply line 74a is arranged within case 100 so as to, for example, make one revolution around the periphery of culture vessel 10.

This case 100, case warming heater 103 and fan 104 compose the aforementioned culture vessel warming unit 93.

In addition, as shown in FIG. 1, an objective lens 110 that detects images, fluorescent intensity and so forth of cells A is arranged below case 100, while a transmitting light source 111 that radiates light onto cells A to acquire images of the cells A by measurement of phase difference or measurement of differential interference and so forth is arranged above case 100. Images of cells A detected with objective lens 110 are recorded as electronic images by PC 120 by means of a CCD camera and so forth not shown. In addition, in the case of observing the expression of a fluorescent label within cells A, together with radiating light of a specific wavelength through objective lens 110, light containing the fluorescent component emitted from cells A is captured with objective lens 110. The fluorescent intensity of only the fluorescence required for examination as obtained with a wavelength-selective filter not shown is converted to a numeric value by a fluorescent intensity detector such as a CCD, photomultiplier or photodiode and recorded in PC 120. Namely, objective lens 110, transmitting light source 111 and PC 120 compose the aforementioned detection device 30.

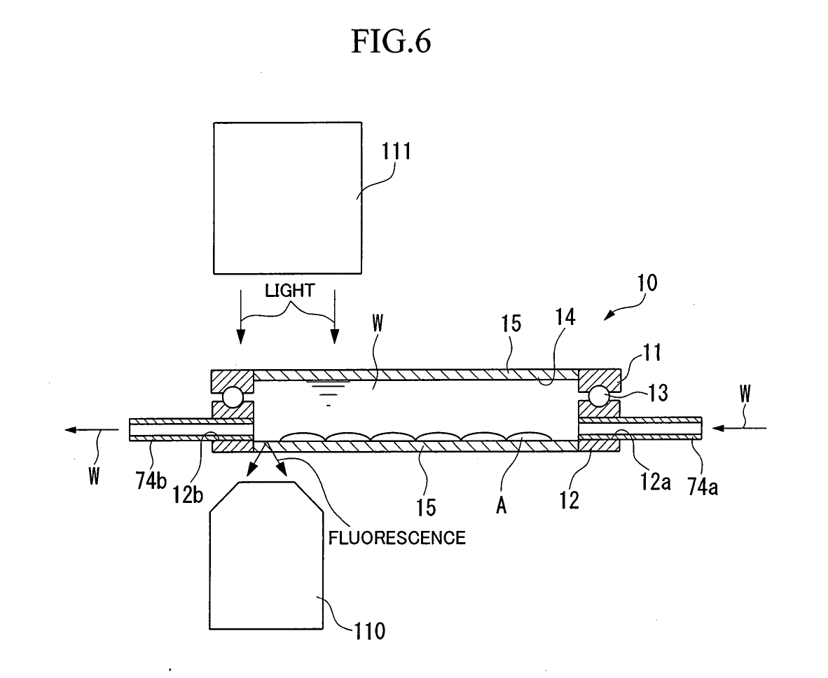

In addition, objective lens 110 and transmitting light source 111 have a function by which they detect a feature such a fluorescent intensity from cells A as previously described as well as a function by which they measure the level of auto-fluorescence of culture liquid W within culture vessel 10. Namely, as shown in FIG. 6, auto-fluorescence is detected by observing culture liquid W filled within culture vessel 10 from below with objective lens 110. In addition, the measured level of auto-fluorescence of culture liquid W is transmitted to PC 120. Namely, this objective lens 110 and transmitting light source 111 compose the aforementioned measuring device 50.

Together with having a function for integrally controlling each of the aforementioned components, the aforementioned PC 120 also has a function that controls the temperature of the aforementioned warming device 90 so as to maintain the temperature of culture vessel 10 at a predetermined temperature such as 37° C. based on the temperature of the culture vessel 10 measured with temperature sensor 73. Namely, PC 120 integrally controls cylindrical heater 91, culture liquid heater 92 and culture vessel warming unit 93.

In addition, PC 120 has the aforementioned discrimination section 60 that judges whether or not culture liquid W has degraded. The discrimination section 60 has a function that judges the degradation of culture liquid W by accumulating the levels of auto-fluorescence of culture liquid W transmitted from the aforementioned measuring device 50, converting them to measured values according to intensity, and comparing them with a preset threshold value. In addition, in the case culture liquid has been judged to have degraded, discrimination section 60 has a function by which, together with automatically replacing or replenishing the culture liquid W inside culture liquid tank 75 by operating tank supply pump 82a and tank discharge pump 83a, supplies fresh culture liquid W to culture vessel 10 by operating syringe piston pump 71. Namely, discrimination section 60, tank supply pump 82a, tank discharge pump 83a, syringe piston pump 71, culture liquid supply line 74a and culture liquid discharge line 74b compose a culture liquid replacement device 125 that automatically replaces or replenishes culture liquid W.

Furthermore, culture liquid tank 75, culture liquid supply line 74a, culture liquid discharge line 74b, both solenoid valves 80 and 81, and carbon dioxide supply line 85 also compose a portion of the aforementioned culturing device 20.

The following provides an explanation of the case of detecting a feature such as fluorescent intensity from cells A using cell images or a fluorescent label with a cell culture detection apparatus 1 composed in this manner.

First, for the initial setup prior to housing culture vessel 10 inside case 100, PC 120 supplies culture liquid W from a culture liquid supply source into culture liquid tank 75 up to the upper surface from supply port 76a by operating tank supply pump 82a.

Culture liquid W retained in culture liquid tank 75 is then warmed to a temperature that is higher than a predetermined temperature (e.g., 37° C.) of culture vessel 10 but not to a degree that damages the composition by culture liquid tank heater 92. In other words, it is set to a higher temperature in consideration of the cooling action that occurs during the course of transferring liquid to culture vessel 10. In addition, carbon dioxide at a predetermined concentration (e.g., 5%) is simultaneously supplied from carbon dioxide supply line 85 into liquid culture tank 75, together with rotating stirrer 86 of stirring unit 72 to uniformly dissolve a predetermined concentration of carbon dioxide in culture liquid W.

Moreover, PC 120 controls the temperature of cylindrical heater 91 so that culture liquid supply line 74a and culture liquid discharge line 74b reach a temperature that is higher than a predetermined temperature (e.g., 37° C.) of culture vessel 10. In other words, the temperature is set to be higher in consideration of cooling action similar to the aforementioned culture liquid tank heater 92.

In addition, PC 120 controls the temperature of case warming heater 103 to a predetermined temperature (e.g., 37° C.) to warm case 100 as well as the air inside case 100 by using heat transfer. Furthermore, a heat-insulating material and so forth may be provided around the outer periphery of case 100 to reduce the cooling action on case 100 from the outside.

Following completion of the aforementioned initial setup, culture vessel 10 that is holding the cells is housed within case 100. When culture vessel 10 is housed within case 100, temperature sensor 73 contacts the outer surface of culture vessel 10, measures the temperature of the culture vessel 10 and transmits the measurement result to PC 120. In addition, PC 120 then rotates ball screw 43 with motor 44 to house culture vessel 10 within light blocking unit 41.

Moreover, PC 120 circulates culture liquid W by operating by integral control piston 71 a of syringe piston pump 71 and both solenoid valves 80 and 81. Namely, culture liquid W circulates by being taken into culture liquid supply line 74a from supply port 76a, supplied from culture liquid supply port 12a to internal space 14 of culture vessel 10, caused to flow from culture liquid discharge port 12b through culture liquid discharge line 74b, and then returned to culture liquid tank 75 from discharge port 76b.

At this time, culture liquid W flows through culture vessel 10 at a low flow rate of, for example, 1 ml/30 min and in a non-pulsating state. Namely, culture liquid W circulates without imparting pressure wave motion to cells A. As a result, even in the case of HEK293 cells or other cells having a low degree of adhesion, the cells can be prevented from detaching without having an effect on adhesion. In addition, cells A grow while dividing or changing in size and shape according to their cell cycle during the course of culturing. Here, in the case pressure wave motion has changed, external force acts on the cell membrane surface. Whereupon, cells A become defensive with respect to the stimulation generated by the external force, thereby resulting in the possibility of cessation of division or changes in shape and so forth. However, in the present embodiment, since culture liquid W circulates without changing the pressure wave motion due to the use of syringe piston pump 71, cells A are able to grow while being suitably supplied with nutrients resulting from circulation culturing while reducing the burden on the cells as described above.

Moreover, since culture liquid W is circulating, proteins and other interactive substances discharged from the cells can be reused without being completely replaced, thereby enabling culturing that maintains the cellular interaction required for growth of individual cells A and making it possible to culture cells A more efficiently.

In addition, when the temperature of culture vessel 10 is received from temperature sensor 73, PC 120 integrally controls the aforementioned culture liquid tank heater 92, cylindrical heater 91, case warming heater 103 and fan 104 based on the temperature to maintain the temperature of culture vessel 10 at a predetermined temperature (e.g., 37° C.). In other words, the temperature of culture vessel 10 is precisely maintained at a predetermined temperature by, for example, either switching only culture liquid tank heater 92 on and off or suitably combining an operation such as setting the temperature of cylindrical heater 91 to a higher temperature and so forth corresponding to a change in the external environmental temperature and predetermined temperature value. As a result, the temperature burden on the cells within culture vessel 10 can be effectively reduced.

In this manner, together with cells A being cultured using a circulation culturing system at the optimum culturing temperature and carbon dioxide concentration within culture vessel 10, as shown in FIG. 5, they are cultured without being affected by phototoxicity in a darkroom state in which they are blocked from indoor light and other environmental light L within light blocking unit 41. Thus, cells A can be cultured for a long period of time without being subjected to a burden.

Here, in the case of detecting a feature of cells A, ball screw 43 is rotated by means of motor 44 by PC 120, and culture vessel 10 is moved from inside light blocking unit 41 to an observation surface of case 100. As shown in FIG. 3, cells A to be observed are positioned directly above objective lens 110 by moving the X-Y stage. Cells A are then irradiated with an excitation light and so forth from transmitting light source 111, and by detecting the fluorescence emitted from the cells as a result of this irradiation with objective lens 110, the fluorescent intensity of cells A can be detected. In addition, cell images and so forth of cells A can also be detected with objective lens 110. Moreover, cell images or fluorescent intensity or other feature can be detected for each cell A within culture vessel 10 by moving the X-Y stage.

Furthermore, when detecting a cell feature, as shown in FIG. 6, background may be measured with objective lens 10 by radiating light at a location away from cells A prior to detecting a feature of cells A followed by detecting the feature of cells A. In this case, since unnecessary background components can be removed from the detected feature of cells A, the feature of cells A can be detected more accurately.

After detecting a feature of cells A, culture vessel 10 is again housed in light blocking unit 41 by culture vessel transport device 42. As a result, since the effects of phototoxicity can be reduced by blocking environmental light L from cells A except for when detecting a feature, the burden on cells A can be reduced and the cells can be reliably cultured for a long period of time. In addition, since culture vessel 10 can be easily moved in and out of light blocking unit 41 with culture vessel transport device 42, a feature of cells a can be detected on a real-time basis as necessary during the course of culturing.

In addition, circulating culture liquid W can be maintained in the optimum state at all times without degrading during culturing of cells A.

Namely, during the initial culturing setup, as shown in FIG. 6, the auto-fluorescent intensity of culture liquid W is measured with objective lens 110, and that result is transmitted to discrimination section 60 of PC 120. The discrimination section 60 then converts the transmitted auto-fluorescent intensity to a measured value corresponding to the intensity and accumulates that value. The auto-fluorescent intensity of culture liquid W is then suitably measured with objective lens 110 over time during the course of culturing of cells A. For example, auto-fluorescent intensity may be measured corresponding to detection of a feature of cells A, or only the auto-fluorescent intensity of culture liquid W may be measured at certain fixed time intervals.

The measured values of auto-fluorescent intensity measured in this manner and accumulated by discrimination section 60 increase over time. In other words, during the course of culturing, cells A discharge cellular interactive substances as well as waste products into culture liquid W. Accompanying this, nutrients in culture liquid W decrease. As a result of this interaction, culture liquid W degrades resulting in an increase in auto-fluorescent intensity.

Discrimination section 60 then compares the transmitted measured values with a preset threshold value, and when a measured value has reached or exceeded that threshold value, culture liquid W is judged to have degraded. When culture liquid W is judged to have degraded, discrimination section 60 operates culture liquid supply pump 82a to replenish culture liquid tank 75 with fresh culture liquid W from a culture liquid supply source. As a result, since fresh culture liquid that has not degraded is mixed with culture liquid W present in culture liquid tank 75, the degradation of culture liquid W is alleviated overall.

In addition, in the case, for example, the transmitted measured value is much larger than the threshold value and that difference is greater than or equal to a set value, discrimination section 60 judges that culture liquid W has degraded considerably, and together with operating culture liquid supply pump 82a to supply fresh culture liquid W, it also operates culture liquid discharge pump 83a to discharge the previously used culture liquid W from culture liquid tank 75 to replace it with the required amount of fresh culture liquid W.

At this time, PC 120 controls culture liquid supply pump 82a and culture liquid discharge pump 83a using a liquid level sensor and so forth not shown so that the level of culture liquid W does not fall below the bottom of supply port 76a. As a result, air bubbles are prevented from being aspirated through supply port 76a to prevent air bubbles from entering culture vessel 10.

As has been described above, in this cell culture detection apparatus 1, together with cells A being able to be maintained at predetermined culturing conditions such as a temperature of 37° C. and carbon dioxide concentration of 5% in culture vessel 10 by culturing device 20, cells A can be cultured by a circulation culturing system that supplies nutrients as necessary. Since culture vessel 10 can be blocked from indoor light and other environmental light L by light blocking device 40 when a feature of cells A is not detected in particular, the effect of phototoxicity resulting from radiation of light is eliminated, and the burden on cells A can be reduced. Thus, a feature such as fluorescent intensity can be measured both accurately and on a real-time basis from cells A by detection device 30 while culturing cells A for a long period of time.

In addition, following completion of culturing, since culture vessel 10 can be easily housed within light blocking unit 41 by culture vessel transport device 42, the effect of phototoxicity can be reduced as much as possible.

In addition, since culture liquid W is circulated by syringe piston pump 71, cells A can be cultured while maintaining cellular interactions required for individual cell growth without completely replacing the interactive substances discharged from cells A. At this time, since the carbon dioxide concentration of culture liquid W can be uniformly maintained at the optimum culture concentration by stirring unit 72, culturing can be carried out while effectively reducing the burden on cells A.

Moreover, since culture liquid W circulates without fluctuating in pressure due to syringe piston pump 71, unnecessary pressure wave motion is not imparted to cells in the culture vessel. As a result, since cells A are prevented from detaching and are not subjected to irritation caused by pressure wave motion, the burden on cells A caused by changes in the pressure of culture liquid W can be reduced.

Moreover, since the temperatures of warming device 90, namely case warming heater 103, fan 104, cylindrical heater 91 and culture liquid tank heater 92, are integrally controlled based on the temperature of culture vessel 10 as measured by temperature sensor 73, culture vessel 10 can be precisely maintained at a temperature of, for example, 37° C., thereby making it possible to reduce the burden on cells A in culture vessel 10 attributable to temperature.

In addition, during the course of culturing cells A, in the case the auto-fluorescent intensity of culture liquid W is measured by culture measuring device 50, and discrimination section 60 has judged that culture liquid W has degraded after assessing the degradation status of culture liquid W based on the auto-fluorescent intensity, the cells can be cultured while maintaining culture liquid W in the optimum state in which it has not degraded by replenishing or replacing culture liquid W with culture liquid replacement device 125. Thus, cells A can be cultured in the optimum culture liquid at all times, and the burden on cells A caused by degradation of culture liquid W can be reduced. In particular, since the replacement and so forth of culture liquid W can be carried out automatically by culture liquid replacement device 125, constant culturing conditions can be maintained at all times.

As has been described above, since culturing can be carried out for a long period of time by reducing the burden on cells A on an X-Y stage, in addition to being able to measure time-based changed in cells A on a real-time basis or easily detect changes and so forth that occur during the course of culturing, the behavior of continuously activated (stabilized) cells A can be accurately detected.

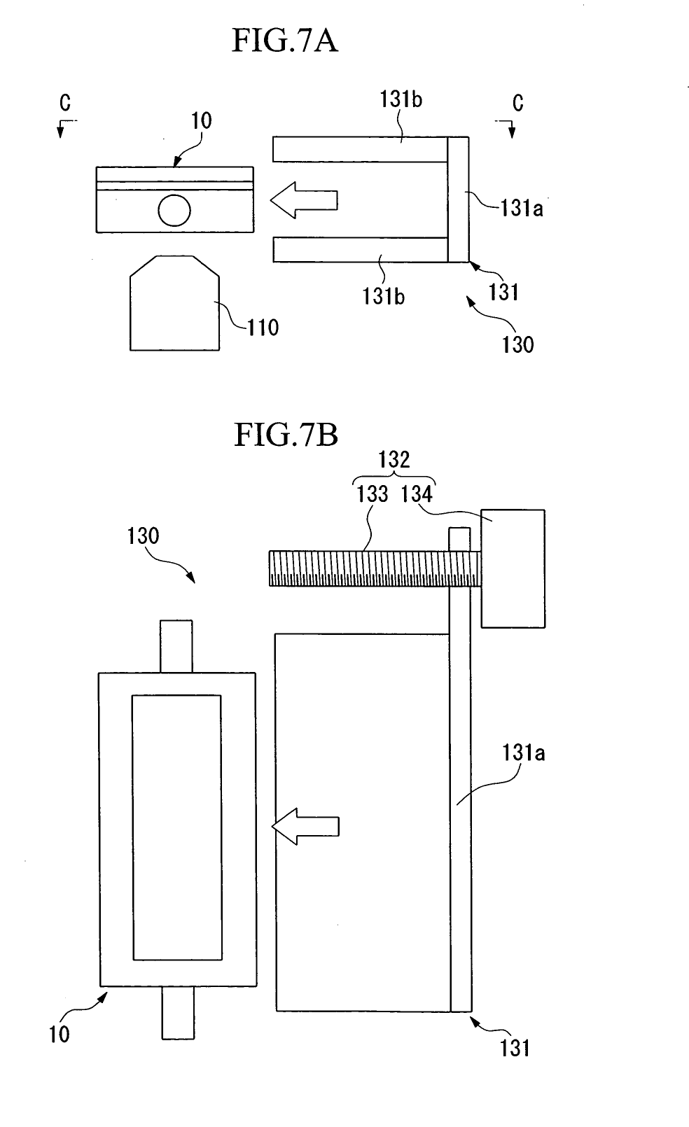

Next, an explanation is provided of a second embodiment of the present invention with reference to FIG. 7. Furthermore, in this second embodiment, those sections that are identical to the constituent features of the first embodiment are indicated with the same reference symbols, and their explanations are omitted.

The second embodiment differs from the first embodiment in that, in contrast to culture vessel 10 being moved in and out of light blocking unit 41 as a result of being moved by culture vessel transport device 42 in the first embodiment, in light blocking device 130 of the second embodiment, culture vessel 10 is blocked from light by moving light blocking unit 131.

Namely, as shown in FIG. 7, light blocking device 130 is provided with a light blocking unit 131 that blocks light from the periphery of culture vessel 10, and light blocking unit transport device 132 that transports light blocking unit 131 around the periphery of culture vessel 10. The aforementioned light blocking unit 131 has a shaft member 131 a and a pair of thin plate members 131b, and one end of thin late members 131b is attached to the top and bottom of shaft member 131a so as to have a U-shaped cross-section of which the opening faces toward culture vessel 10 while having a size that allows it to house culture vessel 10 inside. Furthermore, a sponge or other low-reflecting member may be attached to the inner surface of light blocking unit 131 to enhance the ability to block environmental light L from the outside.

The aforementioned shaft member 131a is locked to a ball screw 133 arranged so as to be perpendicular to the axial member 131a. In addition, the base of the ball screw 133 is rotatably supported by motor 134 fastened within case 100. In addition, In addition, the operation of motor 134 is controlled by PC 120. Namely, this ball screw 133 and motor 134 compose the aforementioned light blocking unit transport device 132. Furthermore, culture vessel 10 is fixed on an observation surface within case 100.

In light blocking device 130 composed in this manner, except for when detecting a feature of cells A, light blocking unit 131 is positioned around the periphery of culture vessel 10 to block environmental light L from cells A. In addition, when detecting a feature of cells A, motor 134 is driven by PC 120 causing ball screw 133 to rotate and move light blocking unit 131 to expose culture vessel 10 after which it can be observed.

In this manner, culture vessel 10 can be easily and reliably blocked from light by light blocking unit transport device 132, thereby making it possible to reduce the effect of phototoxicity on cells A therein. In addition, since it is not necessary to move culture vessel 10, the burden on cells A can be further reduced.

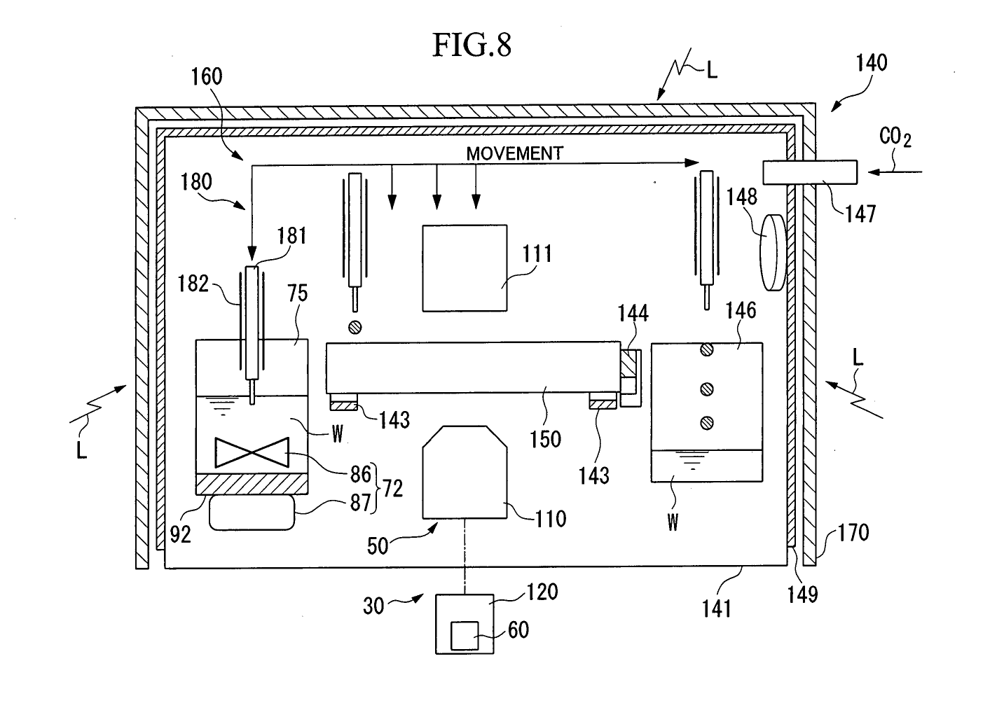

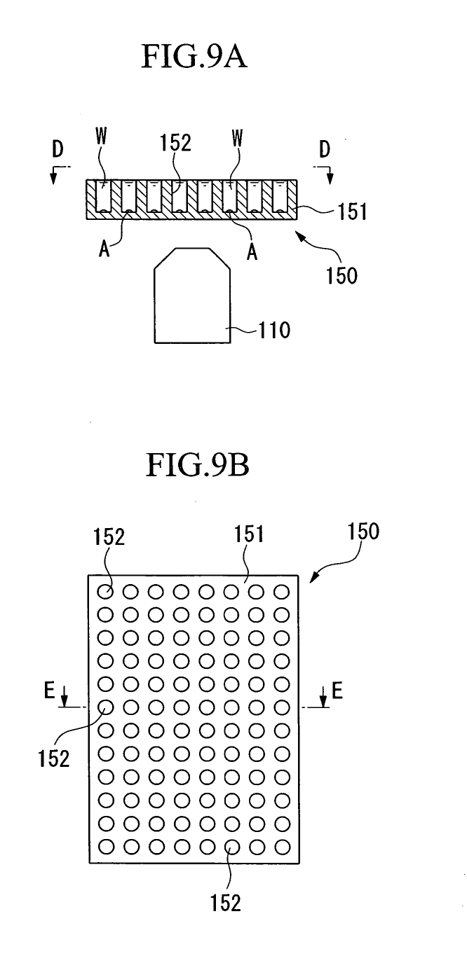

Next, an explanation is provided of a third embodiment of the present invention with reference to FIGS. 8 and 9. Furthermore, in this third embodiment, those sections that are identical to the constituent features of the first embodiment are indicated with the same reference symbols, and their explanations are omitted.

The third embodiment differs from the first embodiment in that, in contrast to cells A being cultured while circulating culture liquid W in the first embodiment, in the case of cell culture detection apparatus 140 of the third embodiment, cells A are cultured without circulating culture liquid W.

Namely, as shown in FIGS. 8 and 9, cell culture detection apparatus 140 is provided with a 96-well microplate (culture vessel) 150 that houses cells A together with culture liquid W, a culturing device 160 that cultures the cells A under predetermined culturing conditions, a case cover (light blocking device) 170 that blocks environmental light L from 96-well microtiter plate 150 when a feature of cells A is not detected from cells A during culturing, a detection device 30 that detects a feature of cells A, a measuring device 50 that measures auto-fluorescence of culture liquid W, and a discrimination section 60 that judges whether or not culture liquid W has degraded based on the measurement results obtained from the measuring device 50.

The aforementioned 96-well microtiter plate 150 is placed on a microscope stage not shown that is arranged in case 141. As shown in FIG. 9, 96 wells 152 are formed separated by roughly 9 mm intervals in a plastic plate 151, and each well 152 is capable of housing cells A and culture liquid W. In addition, the bottom of plate 151 is in the form of a glass member having an optically flat surface, and cells A within each well 152 can be observed from below with objective lens 110.

In addition, as shown in FIG. 8, 96-well microtiter plate 150 is made to be warmed by conduction of heat by a warming heater (culture vessel warming unit) 143 that is one of the warming devices. Furthermore, the temperature of warming heater 143 is controlled by PC 120. Moreover, the temperature of 96-well microtiter plate 150 is measured by temperature sensor 144, and the results of measurement are sent to PC 120.

A culture liquid tank 75 that holds culture liquid W, a culture liquid discharge tank 146 that stores unnecessary culture liquid W, a carbon dioxide supply line 147 that supplies carbon dioxide of a predetermined concentration (e.g., 5%) to case 141 from a carbon dioxide supply source arranged outside, a fan 148 that circulates the internal air within case 141, and a pipetting unit that pipettes culture liquid W are provided within the aforementioned case 141. Moreover, a case warming heater (culture liquid warming unit) 149, which is one of the warming devices for warming the internal space of case 141, is provided around the periphery of the case 141, and its temperature is controlled by PC 120. In other words, together with carbon dioxide of a predetermined concentration being filled within case 141 by the aforementioned carbon dioxide supply line 147, air warmed by case warming heater 149 and fan 148 circulates to maintain the inside of case 141 at a uniform predetermined temperature (e.g., 37° C.). Thus, culture liquid W contained in each well 152 of 96-well microtiter plate 150 is maintained at a state of, for example, a temperature of 37° C. and carbon dioxide concentration of 5%.

The aforementioned culture liquid tank 75 is arranged adjacent to one side of 96-well microtiter plate 150. Culture liquid W retained in this culture liquid tank 75 is warmed by culture liquid tank heater 92, and uniformly contains carbon dioxide of a predetermined concentration by stirring unit 72.

The aforementioned culture liquid discharge tank 146 is arranged so as to be adjacent to the other side of 96-well microtiter plate 150.

The aforementioned pipetting unit 180 is provided towards the top of case 141, and has a pipetting nozzle 181 capable of moving horizontally and vertically within case 141. Namely, together with moving between culture liquid tank 75 and culture liquid discharge tank 146, pipetting nozzle 181 is also able to scan each well 152 of 96-well microtiter plate 150 by moving to each of the wells 150. In addition, pipetting nozzle 181 is able to aspirate and discharge culture liquid W therein, and is able to warm culture liquid W that has been aspirated therein with cylindrical heater (culture liquid warming unit) 182, which is one of the warming devices. The temperature of this cylindrical heater 182 is controlled by PC 120.

Furthermore, pipetting unit 180 has a line, syringe piston pump, solenoid valve, scanning axis unit and forth in addition to pipetting nozzle 181.

The aforementioned case cover 170 is formed of an optically opaque material and of a size that covers case 141 from its periphery, and is able to block environmental light L from the outside to maintain the inside of case 141 in the state of a darkroom.

The aforementioned case 141, culture liquid tank 75, culture liquid discharge tank 146, carbon dioxide supply line 147, fan 148, pipetting unit 180, stirring unit 72, warming heater 143, case warming unit 149, culture liquid tank heater 92 and cylindrical heater 182 compose the aforementioned culturing device 160.

Furthermore, in the present embodiment, in the case discrimination section 60 has judged that culture liquid W has degraded, it is set to automatically replace or replenish culture liquid W by operating pipetting unit 180.

In a cell culture detection apparatus 140 composed in this manner, after housing culture liquid W and cells A in each well 152 of 96-well microtiter plate 150 and placing inside case 141, the periphery of case 141 is covered with case cover 148 by a case cover transport device. Simultaneously, PC 120 integrally controls warming heater 143, case warming heater 149 and fan 148 so that the temperature of 96-well microtiter plate 150 reaches a predetermined temperature (e.g., 37° C.) based on the temperature of the 96-well microtiter plate 150 sent from temperature sensor 144.

As a result, cells A are cultured at a temperature of 37° C., carbon dioxide concentration of 5% and in a darkroom state blocked from environmental light L without being affected by phototoxicity.

In addition, together with warming culture liquid W in culture liquid tank 75 to a temperature that is higher than a predetermined temperature (e.g., 37° C.) but not to a degree that damages the composition by controlling culture liquid tank heater 92 and stirring unit 72, PC 120 causes a predetermined concentration (e.g., 5%) of carbon dioxide to be dissolved. Moreover, PC 120 warms pipetting nozzle 181 to a temperature that is higher than a predetermined temperature (e.g., 37° C.) by controlling the temperature of cylindrical heater 182. In other words, the temperature is set to a higher temperature in consideration of the cooling action that acts during the time the culture liquid W travels to 96-well microtiter plate 150.

Here, in the case of detecting a feature of cells A, case cover 170 is moved from the periphery of case 141 by a case cover transport device. Next, excitation light is radiated from transmitting light source 111 while scanning the microscope stage, and the fluorescent intensity of cells A can be detected by detecting the fluorescence emitted from cells A with objective lens 110. In addition, images of cells A can also be detected.

After detecting a feature of cells A, case cover 170 covers the periphery of case 141 by being moved by the case cover transport device. As a result, since the effect of phototoxicity is reduced by blocking cells from environmental light L except when detecting a feature of cells A, the burden on cells A is reduced and cells A can be reliably cultured for a long period of time.

In addition, during culturing of cells A, culture liquid W can be replenished or replaced corresponding to the degree of degradation of culture liquid W. Namely, discrimination section 60 operates pipetting unit 180 in the case discrimination section 60 has judged that culture liquid W has degraded as a result of the auto-fluorescent intensity of culture liquid W transmitted from objective lens 110 being equal to or exceeding a preset threshold value. When pipetting unit 180 receives a signal from PC 120, it moves pipetting nozzle 181 to culture liquid tank 75 where it aspirates culture liquid W inside. Following aspiration, pipetting nozzle 181 is then moved to a well 152 of 96-well microtiter plate 150 where it discharges the aspirated culture liquid W to replenish the culture liquid inside. As a result, since fresh culture liquid W that has not degraded is mixed within well 152, the degradation of culture liquid W is alleviated overall.

In the case culture liquid W has been judged to have degraded considerably, culture liquid W can also be replaced. Namely, after discharging the degraded culture liquid W within a well 152 into culture liquid discharge tank 145 with pipetting nozzle 181, fresh culture liquid W is discharged from culture liquid tank 75.

Furthermore, since culture liquid W is warmed by cylindrical heater 182 during the time it is aspirated and transported by pipetting nozzle 181, it is maintained at the same predetermined temperature as when it was retained in culture liquid tank 75 until immediately before being discharged into each well 152. Thus, the burden attributable to temperature on cells A can be reduced.

In addition, although the degradation rate of culture liquid W varies in the case of culturing different numbers of cells A or different types of cells A in each well 152 of 96-well microtiter plate 150, in the present embodiment, together with being able to detect the degradation of culture liquid W in each well 152, culture liquid W can be replenished or replaced selectively for each well 152. In other words, cellular interactive substances can be prevented from being easily replaced.

The burden on cells A in case 141 can be reduced and they can be cultured for a long period of time in this cell culture detection apparatus 140. In particular, since different numbers of cells A or different types of cells A can be cultured for a long period of time in each well 152 of 96-well microtiter plate 150, in addition to being able to easily detect time-based changes according to the number of cells or cell type as well as changes that occur during the course of culturing on a real-time basis, the behavior of continuously activated (stabilized) culture cells can be accurately detected.

Furthermore, the technical scope of the present invention is not limited to the aforementioned embodiments, and various alterations can be added provided they are within a scope that does not deviate from the gist of the present invention.

Furthermore, although a stirring unit was employed in the first embodiment to make the carbon dioxide concentration of the culture liquid retained in the culture liquid tank uniform, the present invention is not limited to this, but rather any constitution may be employed that makes the carbon dioxide concentration uniform. For example, a rocking agitation system that agitates the culture liquid by rocking the entire culture liquid tank, or an agitation system such as an ultrasound agitation system that agitates the culture liquid by irradiating with ultrasonic waves, may also be employed.

Moreover, although the discrimination section was made to replenish or replace the culture liquid by operating a culture liquid replacement device when it judged that the culture liquid had degraded, it may also be composed to as to emit an alarm.

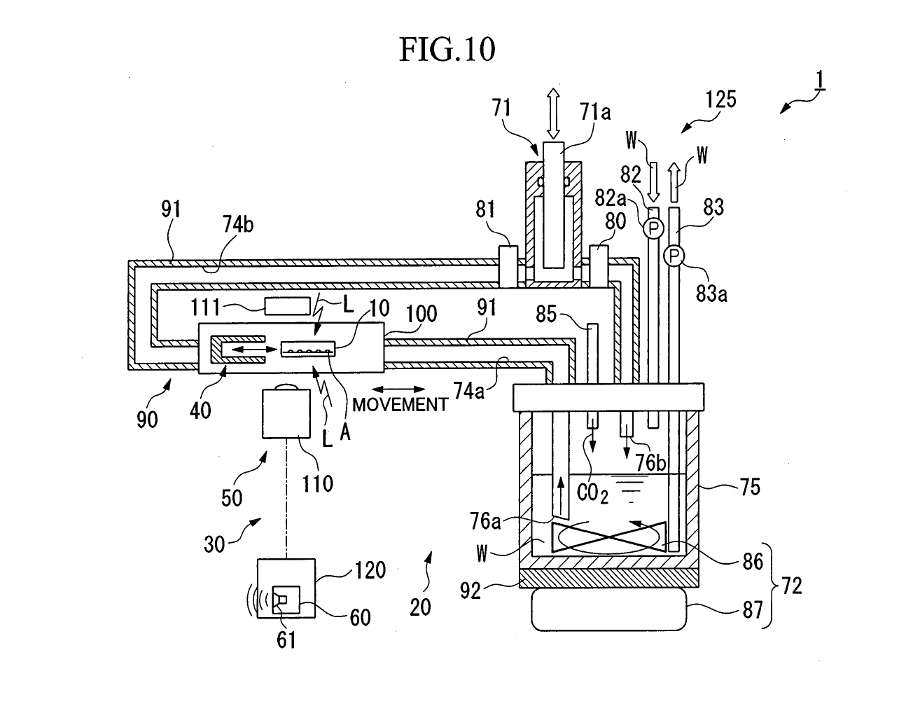

Namely, as shown in FIG. 10, discrimination section 60 has a buzzer that emits an alarm (notification device) when discrimination section 60 has judged that culture liquid W has degraded. As a result, since, for example, an observer is able to accurately and easily be made aware that culture liquid W has degraded as a result of the sounding of buzzer 61, the required processing such as replacement of culture liquid W can be carried out efficiently.

Furthermore, a constitution may also be employed for the cell culture detection apparatus of the first embodiment in which the aforementioned buzzer 61 is simultaneously arranged.

In addition, although a case cover was employed as a light blocking device in the aforementioned third embodiment, the present invention is not limited to this, but rather, for example, a constitution may also be employed in which environmental light is blocked by covering only the 96-well microtiter plate.

In addition, the inside of the case may be made to be maintained at a high humidity in order to prevent drying and evaporation of culture liquid. In addition, the inside of the case may be made to be a sterile environment, and HEPA filters may be provided at those locations where air flows in from the outside.

In the cell culture detection apparatus according to the present invention, together with being able to carry out culturing by the culturing device while maintaining the cells at predetermined culturing conditions of, for example, a temperature of 37±0.5° C. and carbon dioxide concentration of 5%, within the culture vessel, a feature of the cells, such as fluorescent intensity as determined from cell images or by fluorescent labeling, can be measured on a-real-time basis by the detection device while culturing the cells. In addition, since the culture vessel is blocked from indoor light and other environmental light by the light blocking device when the cell feature is not detected, the cells in the culture vessel are not irradiated with light. As a result, since the effect of phototoxicity caused by irradiation with light is decreased, the burden on the viable cells can be reduced. Thus, cells can be reliably cultured in the culture vessel for a long period of time, and a feature such as fluorescent intensity can be measured from the cells during culturing both accurately and on a real-time basis.

In the cell culture detection apparatus according to the present invention, the culture vessel is housed in a light blocking unit in a state in which it is blocked from light except during measurement of a cell feature. In other words, since viable cells in the culture vessel are completely blocked from environmental light by the light blocking unit, the effect of phototoxicity is eliminated and the cells are able to survive for a long period of time. In addition, since the culture vessel can be easily housed within the light blocking unit simply by being moved by a culture vessel transport device following completion of measurement, the effect of phototoxicity can be reduced as much as possible.

In the cell culture detection apparatus according to the present invention, since the culture vessel is housed in a light blocking unit in a state in which it is blocked from light except during measurement of a cell feature, the effect of phototoxicity is eliminated and the cells are able to survive for a long period of time. In addition, since the light blocking unit can easily be positioned around the periphery of the culture vessel simply by being moved by the light blocking unit transport device following completion of measurement, the effect of phototoxicity can be reduced as much as possible. In addition, since it is not necessary to move the culture vessel, the burden on the cells is further reduced.

In the cell culture detection apparatus according to the present invention, since culture liquid within the culture vessel is circulated by a circulation pump, together with being able to carry out culturing in which the cellular interactions required for individual cell growth are maintained without completely replacing the interactive substances discharged from the cells, nutrients can be supplied to the cells as necessary. In addition, the carbon dioxide concentration of the culture liquid can be uniformly maintained at the optimum culture concentration (e.g., 5%) by the stirring unit, and the temperature of the culture vessel can be maintained at the optimum temperature (e.g., 37° C.) as a result of the temperature of the culture liquid being controlled by the culture liquid warming unit. Thus, culturing can be carried out while reliably reducing the burden on the cells.

In the cell culture detection apparatus according to this invention, since culture liquid is pumped without generating pressure fluctuations by a syringe piston pump or other type of non-pulsating circulation pump, the cells in the culture vessel are not subjected to unnecessary pressure fluctuations. As a result, there are no effects on, for example, the detachment of cells or adhesion of cells to the inside of the culture vessel. Thus, the burden on the cells caused by pressure fluctuations in the culture liquid can be reduced thereby enabling long-term culturing.

In the cell culture detection apparatus according to the present invention, since the warming unit controls the temperature based on the temperature measured with a temperature sensor, the temperature of the culture vessel can be precisely maintained at a predetermined temperature (e.g., 37° C.). In other words, the culture vessel is warmed directly by the culture vessel warming unit, the culture vessel is warmed by the culture liquid by warming the culture liquid inside the lines through the supply or discharge line with the line warming unit, or the culture liquid is warmed by warming the culture liquid itself with the culture liquid warming unit. In this manner, the burden on the cells in the culture vessel caused by temperature can be reduced by maintaining the culture vessel at a predetermined temperature. In addition, the culture vessel can be maintained at a predetermined temperature more effectively by suitably combining the culture vessel warming unit, line warming unit and culture liquid warming unit.

In the cell culture detection apparatus according to the present invention, since warm air is blown directly on the outer surface of the culture vessel by the culture vessel warning device based on the temperature measured with the temperature sensor, the temperature of the culture vessel can be maintained at a predetermined temperature such as 37° C. Since the culture vessel is maintained at a predetermined temperature in this manner, the burden on the cells in the culture vessel caused by temperature can be reduced.

In the cell culture detection apparatus according to the present invention, degradation of the culture liquid can be measured with the measuring device while culturing the cells. In other words, the culture liquid begins to degrade over time due to the accumulation of waste products from the cells in the culture liquid during culturing. This degradation is measured as the level of auto-fluorescence. Namely, an increase in the level of auto-fluorescence means that degradation is progressing. Accordingly, by comparing the level of measured auto-fluorescence with, for example, a threshold value with the discrimination device, a judgment can be made as to whether or not the culture liquid has degraded. Thus, since cell culturing can be carried out while quantitatively judging degradation of the culture liquid during the course of culturing, the burden on the cells caused by degradation of the culture liquid can be reduced, thereby enabling long-term culturing.

In the cell culture detection apparatus according to the present invention, cell culturing can be carried out while automatically maintaining the optimum state in which there is no degradation of culture liquid with the culture liquid replacement device. Thus, cells can be cultured in the optimum culture liquid at all times, thereby making it possible to reduce the burden on the cells caused by degradation of the culture liquid.

In the cell culture detection apparatus according to the present invention, notification of degradation of the culture liquid can be made accurately and easily during cell culturing by the notification device, and the required treatment such as replacement of the culture liquid can be carried out efficiently. Thus, the cells can be cultured in the optimum culture liquid at all times, thereby making it possible to reduce the burden on the cells caused by degradation of the culture liquid.

As has been explained above, according to the cell culture detection apparatus according to the present invention, together with being able to culture cells while maintaining predetermined culturing conditions within a culture vessel with a culturing device, a cell feature can be detected by a detection device while culturing the cells. In addition, since the culture vessel is blocked from environmental light by a light blocking device, the effect of phototoxicity caused by irradiation with light can be decreased and the burden on viable cells can be reduced. Thus, the cells can be reliably cultured for a long period of time within the culture vessel, and a feature such as fluorescent intensity can be accurately measured from the cells during culturing on a real-time basis.

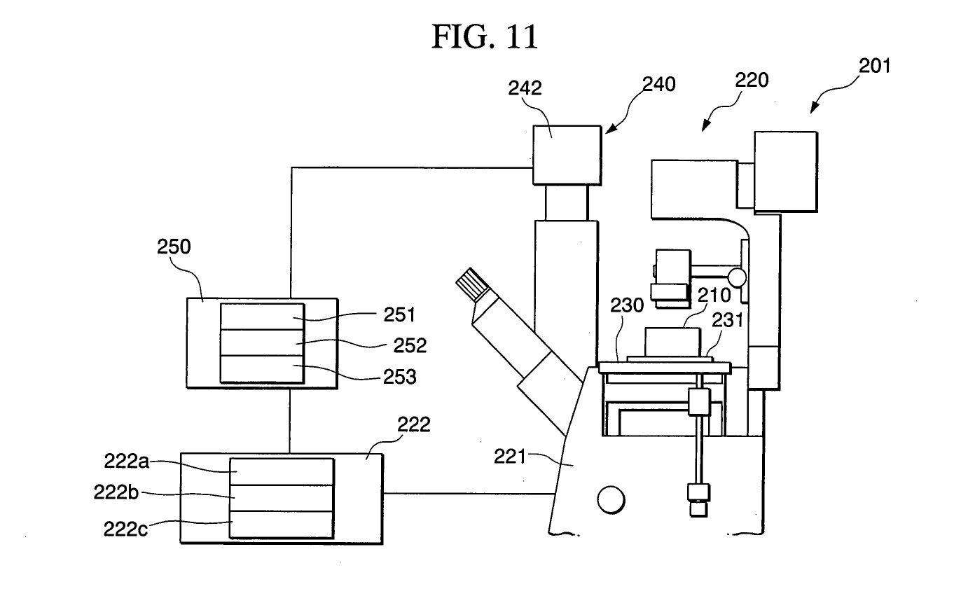

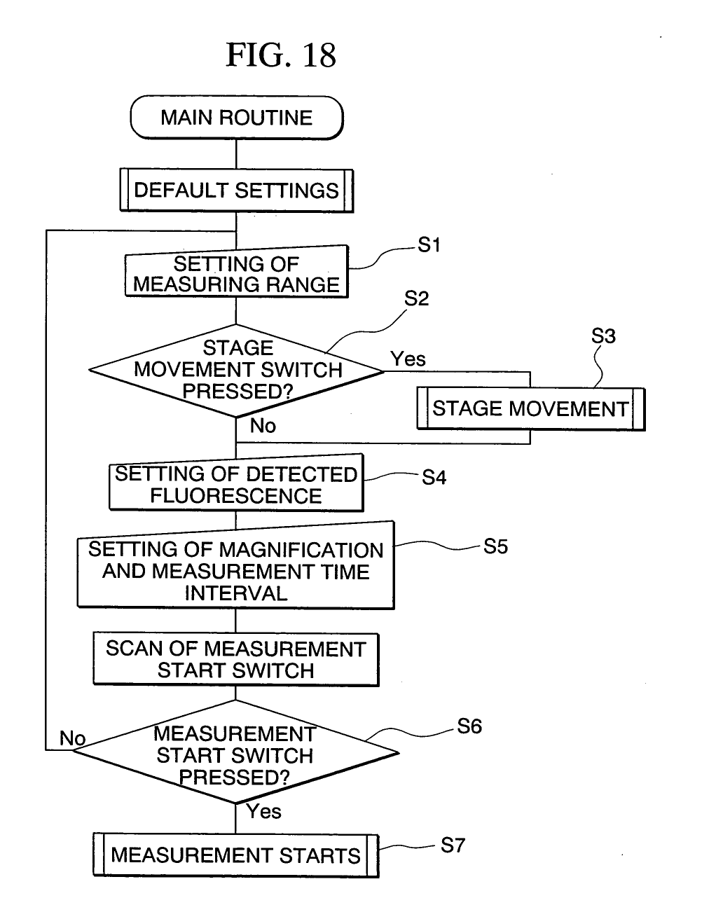

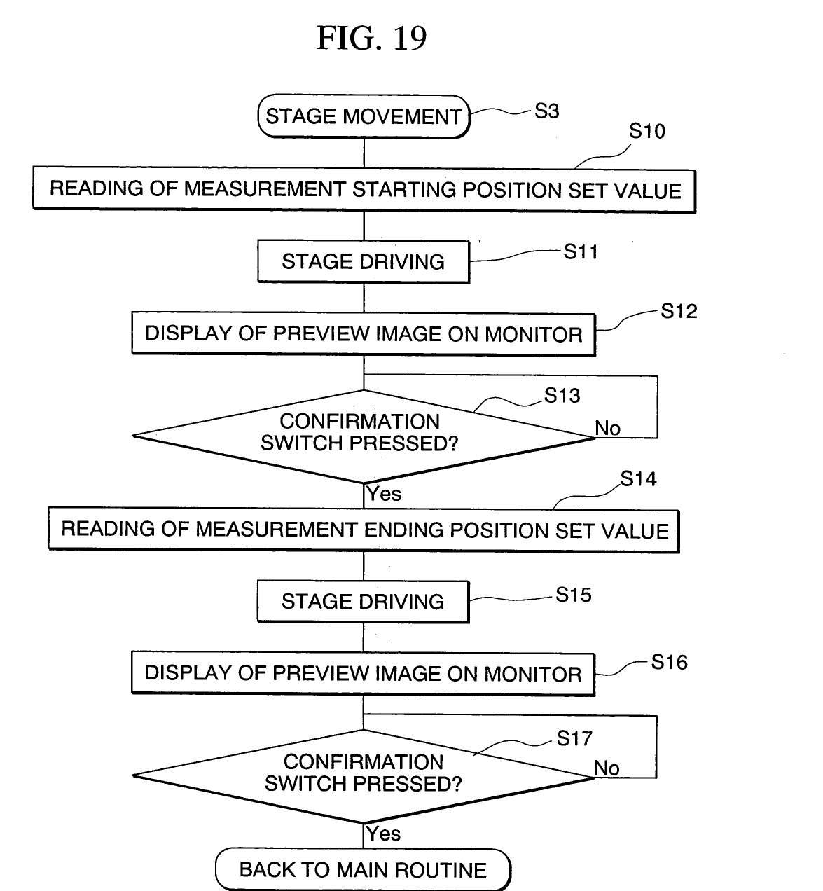

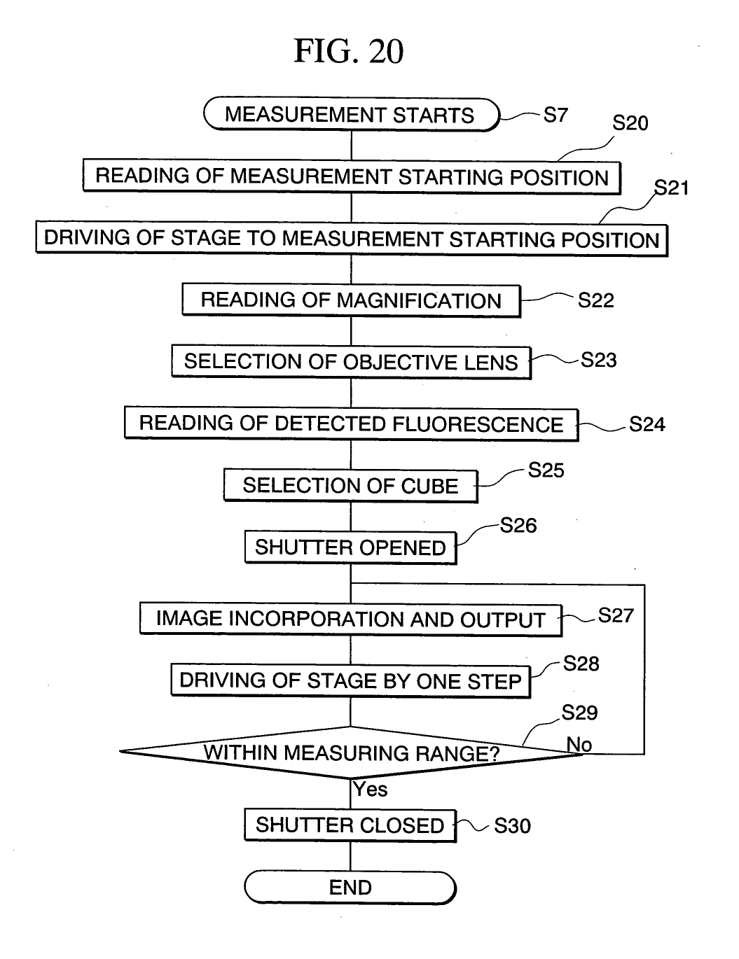

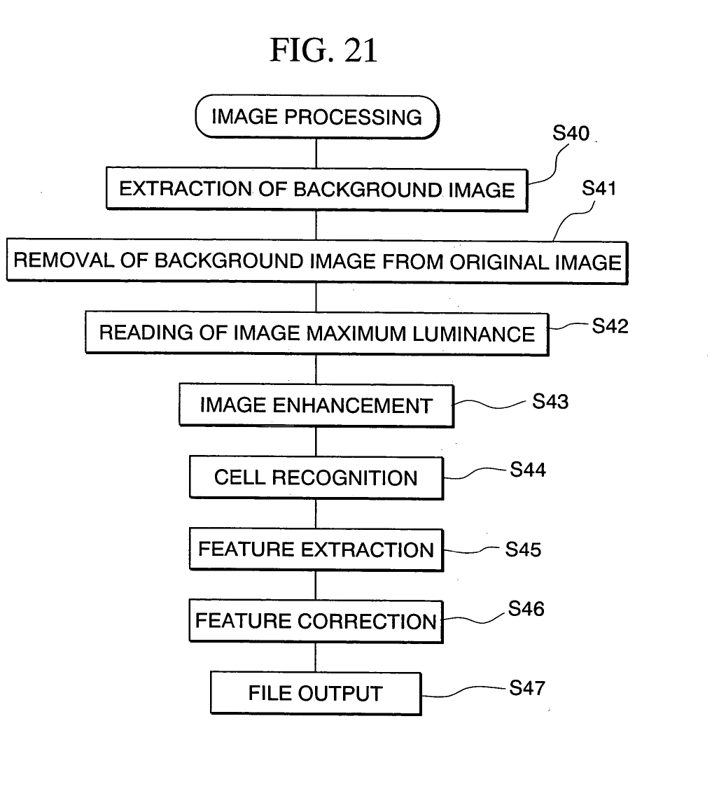

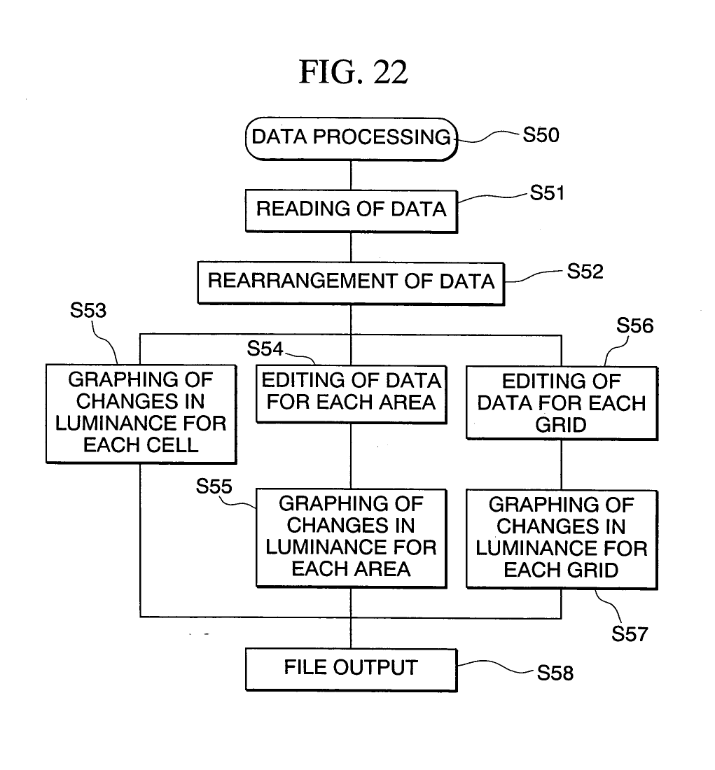

The following provides an explanation of one embodiment of a cell culture observation apparatus 201 according to the present invention with reference to FIGS. 11 to 22.

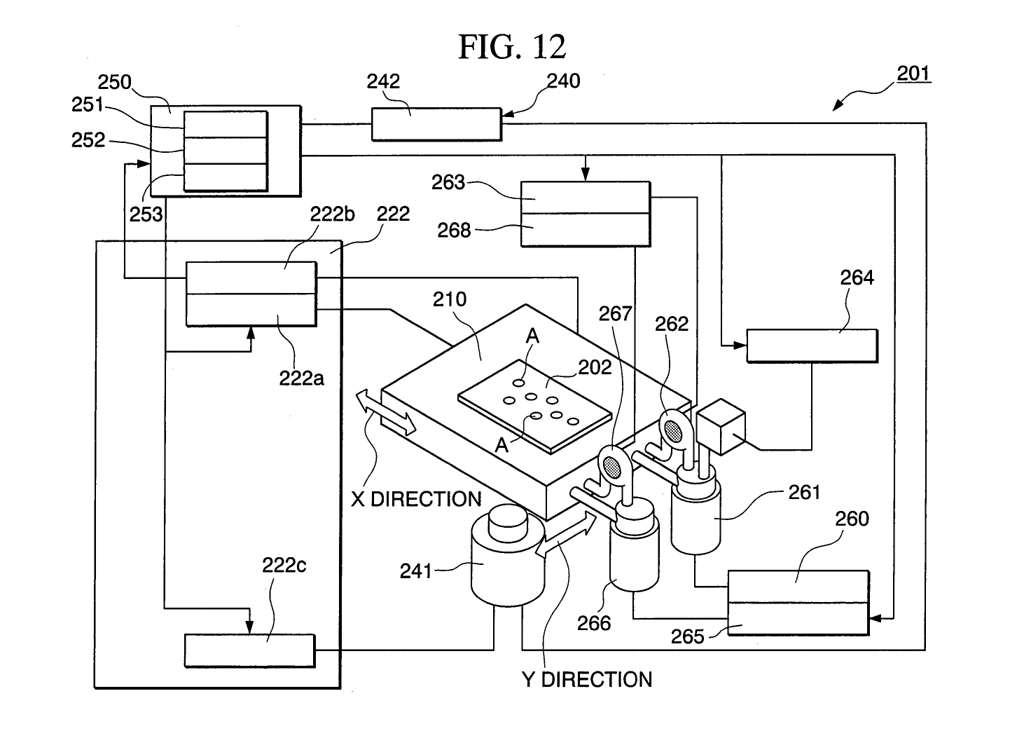

Cell culture observation apparatus 201 of the present embodiment is an apparatus for continuously observing time-based changes in a plurality of cells A present in on a slide glass (support) 202 shown in FIG. 12. Namely, cell culture observation apparatus 201 as shown in FIG. 11 and FIG. 12 is provided with a culture vessel 210, which houses the aforementioned slide glass 202 therein and is capable of maintaining the cellular activity of cells A on the slide glass 202, and an inverted microscope 220 capable of holding the culture vessel 210.

Furthermore, a substrate made of a polymer material and so forth for fixing cells A is treated and formed for use as slide glass 202 of the present embodiment.

The aforementioned inverted microscope 220 has a motorized stage (movable stage) 30 that holds culture vessel 210, and an imaging mechanism (imaging section) 240 that captures images of cells A within culture vessel 210 by dividing into each region corresponding to each cell A.

In addition, cell culture observation apparatus 201 is provided with a personal computer (PC) (analysis section) 250 that analyzes cells A by extracting at least one of either a geometrical feature or optical feature of cells A based on images captured by the imaging mechanism 240.

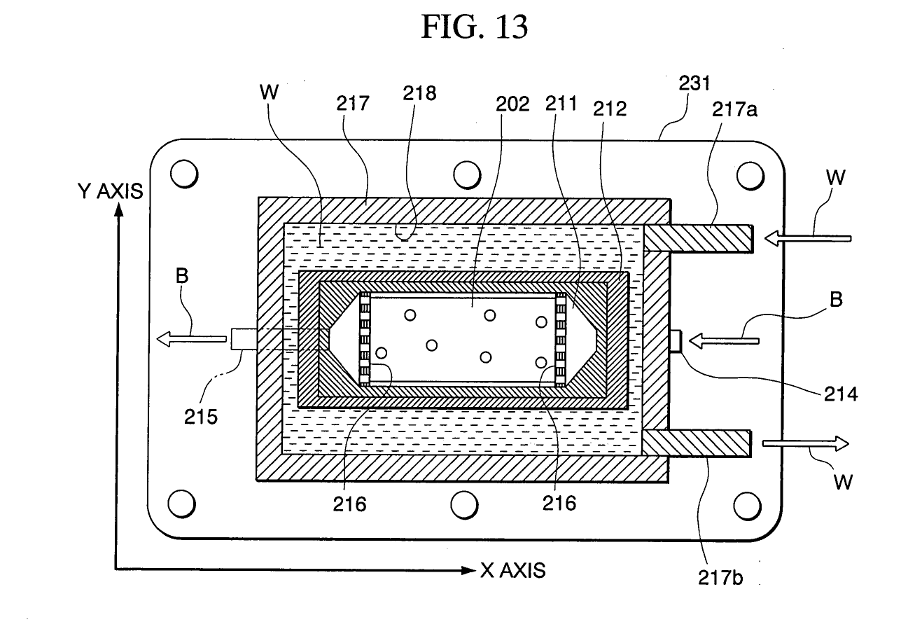

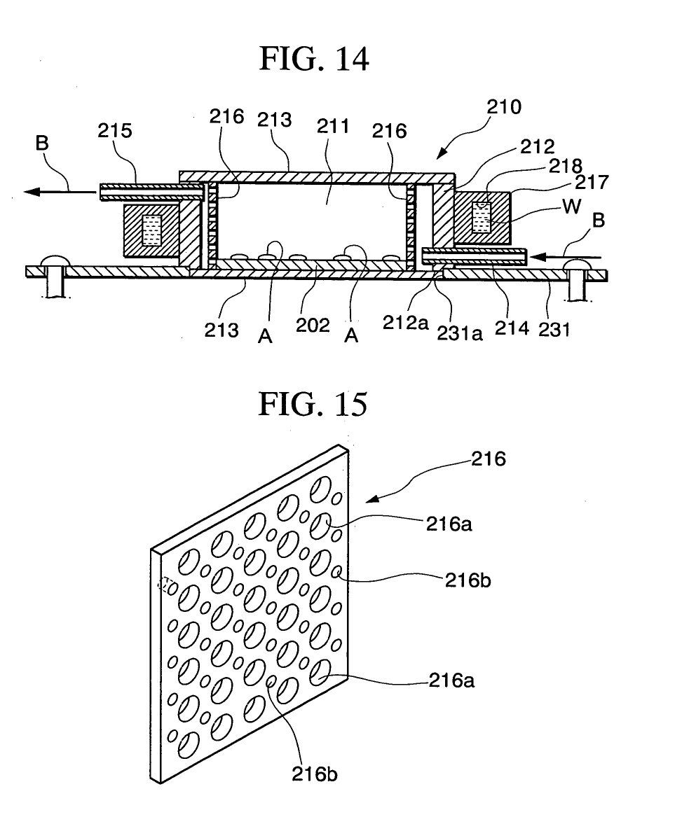

As shown in FIGS. 11 and 12, the aforementioned motorized stage 230 is driven by a motor not shown, and is supported while being able to move in the X and Y directions (horizontal direction) by main frame 221. In addition, a culture vessel mounting section 231 for fixing the aforementioned culture vessel 210 is provided while being able to be adjusted for its level angle (inclination) on this motorized stage 230. Namely, as shown in FIGS. 13 and 14, culture vessel mounting section 231 is formed in the shape of a flat plate, and is fastened to motorized stage 230 by peripheral mounting screws. At this time, inclination can be adjusted relative to the horizontal plane of motorized stage 230 by adjusting the amount by which each mounting screw is tightened. In addition, culture vessel 210 is fixed in culture vessel mounting section 231 by fitting in locking opening 231 a formed in the center of culture vessel mounting section 231. Thus, together with the horizontal plane of culture vessel 210 being able to be adjusted by means of culture vessel mounting section 231, it can be moved in the X and Y directions by means of motorized stage 230.

Moreover, as shown in FIG. 12, an objective lens 241 for observing cells A on slide glass 202 housed in culture vessel 210 is arranged below motorized stage 230, and images captured with the objective lens 241 are made to be output to a CCD camera 242 arranged above main frame 221. Namely, this objective lens 241 and CCD camera 242 compose the aforementioned imaging mechanism 240. In addition, CCD camera 242 has a function which outputs captured images to the aforementioned PC 250 by means of an interface not shown. Furthermore, objective lens 241 has a plurality of lenses with different magnifications, and a desired lens can be selected by changing the lens by turning a revolver not shown.

In addition, as shown in FIGS. 11 and 12, inverse microscope 220 is provided with a microscope control apparatus 222, and a motorized shutter and motorized fluorescent mirror unit not shown. This microscope control apparatus 222 has an X-Y scanning control section 222a, which controls the operation of motorized stage 230, a coordinate detection section 222b, which detects the coordinates of motorized stage 230, and a parallel beam light source control section 222c, which controls the light radiated onto cells A. In addition, coordinate detection section 222b has a function that outputs the detected detection values to the aforementioned PC 250.

The aforementioned PC 250 integrally controls microscope control apparatus 222, namely X-Y scanning control section 222a and parallel beam light source control section 222c. In addition, PC 250 has an image memory section 51, which accumulates captured images that have been sent from imaging mechanism 240, an image processing section 252 that performs image processing (to be described in detail hereinafter) on captured images that have accumulated in the image memory section 51 to analyze them, and a data processing section 53 that detects time-based changes in cells A based on data processed by the image processing section 252. This image processing section 252 and data processing section 253 function corresponding to the desired method by which cells A are observed.

As shown in FIGS. 13 and 14, the aforementioned culture vessel 210 is provided with a rack 212, which together having a through hole 211 capable of housing slide glass 202, is formed from a material such as stainless steel or aluminum having superior thermal conductivity, and a pair of optically smooth glass plates 231a that cover through hole 211 of rack 212 from above and below. A locking section 212a capable of fitting into locking opening 231a of culture vessel mounting section 231 is formed on the lower end of rack 212, and fastened to culture vessel mounting section 231 as a result of the locking section 12a fitting into locking opening 231a.

In addition, gaskets and so forth made of a fluororesin such as tetrafluoroethylene is arranged on the joining surfaces between rack 212 and the pair of glass plates 213 to ensure that the inside is watertight. As a result, since slide glass 202 is housed within culture vessel 210, even if the pair of glass plates 213 are removed from rack 212 and then reattached, the inside of culture vessel 210 can be maintained in a watertight state.

Furthermore, the inner surfaces of the pair of glass plates 213 should be treated to be highly hydrophilic to prevent adherence of air bubbles of culture liquid B. In addition, after housing slide glass 202 within culture vessel 210, the pair of glass plates 213 may be completely sealed and fastened to rack 212 with silicon adhesive and so forth.

In addition, culture vessel 210 is provided with a culture liquid supply pipe 214 for supplying culture liquid B inside rack 212, a culture liquid discharge pipe 215 for discharging culture liquid B that is no longer necessary from inside rack 212, and a pair of flow straightening members 216 for dispersing the flow of culture liquid B.

Culture liquid supply pipe 214 is provided on one end and towards the bottom of rack 212, while culture liquid discharge pipe 215 is provided on the other end and towards the top of rack 212. Namely, after culture liquid B that has been supplied from culture liquid supply pipe 214 has filled the inside of culture vessel 210, it is discharged from culture liquid discharge pipe 215. In addition, as shown in FIG. 12, culture liquid supply pipe 214 is connected to a culture liquid bottle 261 in which the culture liquid temperature is controlled by a culture liquid temperature control section 260. In addition, a culture liquid pump 262 is interposed between culture liquid supply pipe 214 and culture liquid bottle 261, and temperature-controlled culture liquid B is supplied from culture liquid bottle 261 into culture vessel 210 by driving the culture liquid pump 262. This culture liquid pump 262 is, for example, a peristaltic pump or other circulating pump, and the timing of its intermittent driving and flow volume, etc. are controlled by a culture liquid pump control section 263.

In addition, the carbon dioxide concentration of culture liquid B housed within culture liquid bottle 261 is controlled by controlling the flow volume and intermittent supply timing, etc. of carbon dioxide by a carbon dioxide concentration control section 264 so as to maintain a predetermined pH.

As shown in FIGS. 13 and 14, the pair of flow straightening members 216 are arranged within rack 212 between culture liquid supply pipe 214, culture liquid discharge pipe 215 and slide glass 202.

This pair of flow straightening members 216 are formed from plate-shaped porous members having a plurality of through holes in the direction of thickness. Namely, as shown in FIG. 15, together with through holes 216a having a diameter of about 0.1 mm being arranged in the form of a lattice at 0.3 mm intervals, through holes 216b having a diameter of about 0.03 mm are formed in the center at 0.3 mm intervals in the direction of thickness in each flow straightening member 216. In this manner, flow straightening members 216 have two types of through holes 216a and 216b having different diameters.

As a result, as shown in FIGS. 13 and 14, flow straightening member 216 on the side of culture liquid supply pipe 214 is able to distribute culture liquid B supplied to culture vessel 210 from culture liquid supply pipe 214 by dispersing among a plurality of through holes 216a and 216b. In addition, the flow straightening member 216 on the side of culture liquid discharge pipe 215 is able to distribute culture liquid B that is discharged from culture vessel 210 to the outside through culture liquid discharge pipe 215 by dispersing among a plurality of through holes 216a and 216b. Thus, the convergent flow of culture liquid B can be converted into a dispersed flow, thereby enabling culture liquid B to flow at a constant flow rate and flow volume over nearly the entire cross-sectional surface area of culture vessel 210 in the vicinity of slide glass 202 on which cells A are arranged.

In particular, since flow straightening members 216 have through holes 216a and 216b of different diameters, the stagnant flow generated downstream from flow straightening member 216 due to the outflow of culture liquid B from large diameter through holes 216a can be agitated by the outflow from small diameter through holes 216b. Thus, culture liquid B is able to be steadily discharged to the outside from culture vessel 210 by being distributed without becoming stagnant, thereby making it possible to replace culture liquid B.

Furthermore, microscopic through holes having a diameter of, for example, about 0.2 μm may also be employed for the through holes of flow straightening members 216. In this case, the generation of contaminants using culture liquid B as a flow path can also be prevented.

In addition, a temperature control unit 217 is attached to culture vessel 210. This temperature control unit 217 forms a warm water circuit 218 that supplies warm water W around culture vessel 210 inside, and has a warm water supply pipe 217a that supplies warm water W to warm water circuit 218, and a warm water discharge pipe 217b that discharges warm water W from warm water circuit 218. As a result, warm water W can be circulated within warm water circuit 218, thereby making it possible to transfer the heat of warm water W to culture liquid B inside culture vessel 210 through rack 212 of culture vessel 210.

In addition, as shown in FIG. 12, warm water supply pipe 217a is connected to a warm water bottle 266 for which the temperature is controlled by a warm water temperature control section 265. In addition, a warm water pump 267 is interposed between warm water supply pipe 217a and warm water bottle 266, and temperature-controlled warm water W is supplied inside warm water control unit 217 by driving this warm water pump 267. In addition, warm water pump 267 is a peristaltic or other circulating pump, and the timing of its intermittent operation and its flow volume and so forth are controlled by warm water control section 268.

Moreover, warm water control section 268 has a function that controls the temperature and circulating flow volume of warm water W so as to maintain the temperature of culture vessel 210 within the range of 37±0.5° C. by a temperature sensor not shown in the form of, for example, a thermocouple, thermistor or resistance bulb. Thus, culture vessel 210 is able to maintain culture liquid B at a constant temperature without causing sudden changes in temperature as a result of overheating as in the case of a water bath.

The aforementioned culture liquid pump control section 263, carbon dioxide concentration control section 264 and warm water pump control section 268 are integrally controlled by being connected to PC 250. In addition, in order to ensure sterility for cells A, those locations relating to the flow path of culture liquid B, including the inside of culture vessel 210, culture liquid bottle 261, culture liquid pump 262, culture liquid supply pipe 214 and culture liquid discharge pipe 215, are composed to be able to be sterilized.

The following provides an explanation of the case of observing a cell culture with cell culture observation apparatus 201 composed in this manner with reference to FIG. 12 and FIGS. 16 to 22.

First, cell culture observation apparatus 201 is set to an initial state. Namely, as shown in FIG. 12, culture liquid pump control section 263, carbon dioxide concentration control section 264 and warm water pump control section 268 are operated, and together with supplying culture liquid B to culture vessel 210, cell culture observation apparatus 201 is set to predetermined values of, for example, a temperature of 37±0.5° C. and a carbon dioxide concentration of 5%. In addition, together with adjusting culture vessel mounting section 231 so that the entire surface of slide glass 202 within culture vessel 210 is contained within the depth of focus of objective lens 241, the inclination of culture vessel mounting section 231 is adjusted so that slide glass 202 is perpendicular to an optical axis not shown to position slide glass 202.

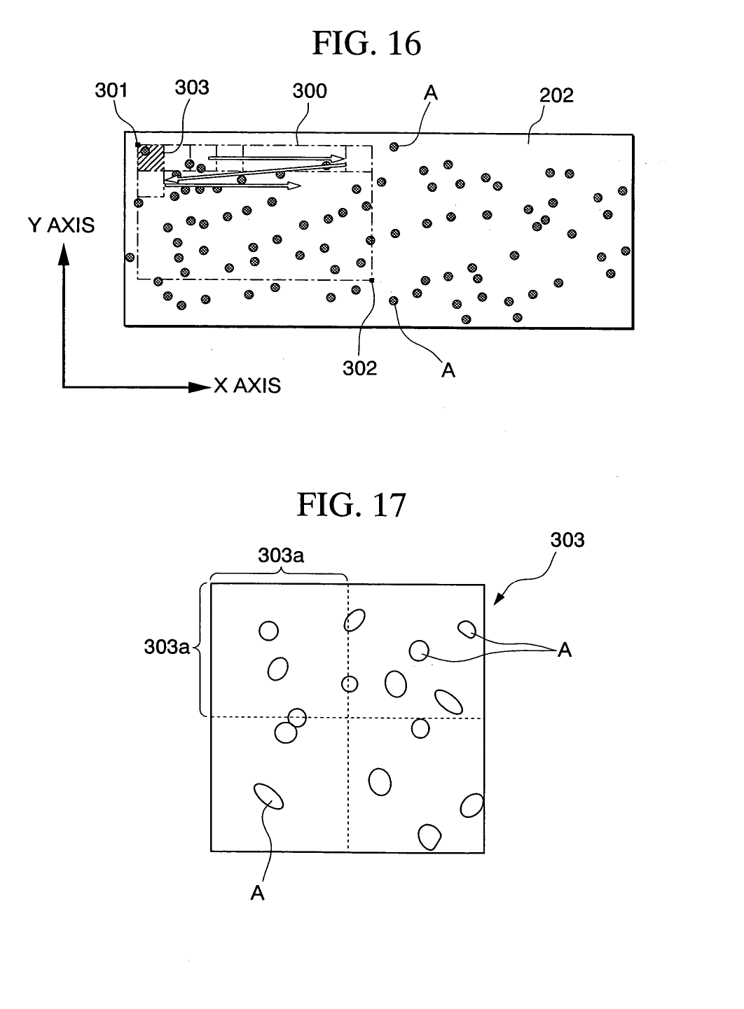

After achieving the aforementioned initial state, as shown in FIG. 16, an operator selects a measuring range 300 of slide glass 202 that is desired to be observed (SI). Namely, the operator inputs values into PC 250 for the coordinate positions of measurement starting position 301 and measuring ending position 302 by using a corner (e.g., the lower left corner as viewed on the paper) of slide glass 202 as the origin. As a result, the range surrounded by both positions 301 and 302 is recognized as the aforementioned measuring range.

Next, the operator operates motorized stage 230 by pressing a stage movement switch not shown on PC 250 (S2) to confirm whether or not the input measuring range 300 is the desired range. Namely, in the case of YES in response to whether or not the stage movement switch has been pressed (S3), together with PC 250 reading the input measurement starting position (S10), motorized stage 230 is moved so that the measurement starting position 301 is positioned within the viewing field of objective lens 241 (S11). When motorized stage 230 moves, a preview screen of measurement starting position 301 is displayed on a monitor not shown of PC 250 (S12). The operator then confirms that slide glass 202 is positioned in the vicinity of measurement starting position 301, while also confirming by viewing the preview screen that a clear image of slide glass 202 is obtained.

After having confirmed measurement starting position 301, the operator then presses a confirmation switch not shown of PC 250 (S13). When this confirmation switch is pressed (case of YES), together with PC 250 reading the input measurement ending position 302 (S14), motorized stage 230 is moved so that measurement ending position 302 is positioned within the field of view of objective lens 241 (S15). When motorized stage 230 moves, a preview screen of measurement ending position 302 is displayed on a monitor not shown of PC 250 (S16). The operator then presses a confirmation switch after judging that measurement ending position 302 is appropriate in the same manner as previously described (S17).

As a result, even if the coordinate positions of measurement starting position 301 and measurement ending position 302 are input incorrectly by the operator, they can be judged prior to the start of measurement. In this case, namely in the case of NO with respect to pressing the confirmation switch, the operator is able to re-input the coordinate positions of measurement starting position 301 and measurement ending position 302 to obtain the desired measuring range 300.

Following completion of setting the measuring range 300, the operator selects the fluorescent protein to be used, such as GFP or HC-Red that has been preliminarily stored in PC 250 (S4). PC 250 then automatically selects the optimum cube (optical filter) for the selected fluorescent protein. As a result, the desired fluorescent light can be detected from cells A. Furthermore, this setting is not limited to being performed once, but may be performed a plurality of times. Multi-color measurement can be carried out by using multiple settings. This is particularly effective in the case of detecting multiple types of proteins from cells A in a single observation. In addition, the cube used during measurement is changed automatically in synchronization with the driving of motorized stage 230.

Following completion of setting of the fluorescent protein, the operator then sets the measurement magnification and measurement time interval (S5). After completing all of the aforementioned settings, the operator presses a measurement start switch not shown on PC 250 (S6) to begin the imaging step. Furthermore, in the case of desiring to change any of the aforementioned settings, namely in the case of NO when the measurement start switch is not pressed, each setting can be reset starting with the setting of measuring range 300.

When the measurement start switch is pressed (case of YES) (S7), together with reading the input viewing field of objective lens 241 (S20), PC 250 moves motorized stage 230 so that measurement starting position 301 is positioned in the field of view of objective lens 241 (S21). PC 250 then changes the revolver corresponding to the set measurement magnification (S22) and then selects an objective lens 241 of the desired magnification (S23). Next, PC 250 controls parallel beam light source control section 222c corresponding to the set fluorescent protein (S24) to select the optimum cube (S25).

Next, after opening the shutter (S26), imaging mechanism 240 captures an image of the amount of fluorescent light of cells A corresponding to the wavelength of the selected fluorescent protein and outputs that image to PC 250 (S27). When the captured image is incorporated, X-Y scanning control section 222a inches motorized stage 230 towards the X direction. Namely, X-Y scanning control section 222a inches motorized stage 230 to the next step by defining as one step the measuring field 303 determined by objective lens 241 and CCD camera 242 (S28). When motorized stage 230 is moved by one step, imaging mechanism 240 captures the image of measuring field 303. In this manner, images are continuously captured towards the X direction while repeating imaging and one-step movement. When scanning in the X direction in measuring range 300 has been completed, X-Y scanning control section 222a, after having received an end flag, scans one step by moving motorized stage 230 towards the Y direction and then again performs scanning towards the X direction. Measurement is then carried out until measurement end position 302 is reached by repeating the aforementioned process (S29). In the case of YES when capturing of images of the entire range of measuring range 300 in this manner has been completed (S30), the shutter is closed and imaging by imaging mechanism 240 and movement by motorized stage 230 stop.

On the other hand, images that have been captured for each step, namely by dividing into each measuring field 303 corresponding to each cell, are sent to PC 250 and accumulated in image memory section 51. Image processing section 252 then recognizes measuring field 303 and the coordinate positions of each cell A within the measuring field 303 based on the captured images accumulated in image memory section 251. In addition, at this time, image processing section 252 calculates and extracts the location of the center of gravity, surface area and other geometrical features as well as fluorescent luminance and other optical features according to the analysis step. As a result, since image processing section 252 accurately extracts the features of each cell A, each cell A is identified and analyzed by accurately correlating with this positional information.

Thus, image processing section 252 is able to convert into images the distribution of fluorescence and so forth of cells A at each position over the entire surface of slide glass 202. In addition, the distribution of fluorescence and so forth can also be converted into images by focusing on each measuring field 303. In addition, since image processing section 252 is able to accurately track each cell A, for example, attention can be focused only on an arbitrary number of cells A, and the distribution of fluorescence within the cells A can be measured locally over a long period of time while culturing. As another example, the amount of fluorescence of each cell A relative to elapsed time can be measured automatically by measuring the entire surface of slide glass 202 at constant time intervals while culturing cells A.

Furthermore, in the case of specifying a plurality of fluorescent proteins and magnifications, PC 250 automatically changes objective lens 241 and the cube following measurement of measuring range 300, and performs measurement by performing operations similar to those described above. In this case, since image processing section 252 extracts luminance for each wavelength of cells A corresponding to a plurality of fluorescent proteins, numerous types of proteins and so forth can be observed in a single observation.

Furthermore, in the case the focus shifts when changing objective lens 241, this should be accommodated by inter-object parfocal correction or an auto-focusing mechanism.

In addition, cell culture observation apparatus 201 of the present embodiment is able to culture cells A for a long period of time and detect time-based changes of a single cell A.

In this case, extraction of the location of the center of gravity, surface area or other geometrical feature or luminance or other optical feature of each cell A is extracted with higher precision. Namely, together with image processing section 252 extracting background images from the captured images accumulated in image memory section 251 (S40), it removes the background from the original captured images (S41). The images are then enhanced so that each cell can be easily recognized in particle form from the images from which background has been removed. In other words, after reading the maximum luminance range of the images that can be enhanced (S42), the images are enhanced by, for example, applying a predetermined value corresponding to this (S43). By then extracting images equal to or greater than a threshold value, for example, from the enhanced images, the individual luminance of each cell A can be recognized in the form of well-defined particles (S44).

As a result, together with more accurately recognizing the geometrical features or optical features of each cell A, they are extracted by correlating with the positional information of cells A (S45). Following extraction, a correction is made for the enhancement work performed to recognize cells A (S46). The corrected features are then, for example, output to a file and accumulated in the file (S47).

Furthermore, the aforementioned enhancement of captured images may also be carried out by recognizing the cell portions from binary images having several binary levels, and using them as masks of the original captured images. In addition, cells may also be recognized by enhancing only the edges of cell luminance and using those edges as a reference. Moreover, a method may also be employed in which the cell portions are recognized by converting clarified images into binary values and then using them as masks of the original captured images.

In addition, a method may be employed for removing the background in which luminance equal to or greater than a fixed level is flattened. Moreover, although the captured images may be discarded since the size of the data becomes quite large, successively storing the images allows them to be used when repeating calculations.

Next, data processing is performed on the features of cells A accumulated in the aforementioned file by data processing section 253 (S50). First, data processing section 253 reads the features accumulated in the field (S51), and then rearranges them in a time series for each cell A (S52). After rearranging the data, data processing section 253 graphs the time-based changes in the differences in luminance, namely expression level, for each cell A (S53).

At this time, the data of cells A for each grid shown in FIG. 17, namely measuring field 303, can be edited (S54) to graph the time-based changes in luminance as necessary (S55).

Moreover, the data of cells A for each area 303a shown in FIG. 17, namely the range over which measuring field 303a is divided up more finely, can also be edited (S56) to graph the time-based changes in luminance (S57). Furthermore, area 303a is arbitrarily set by the operator. In addition, those cells A which are present on a border of the grid or an area 303a are judged according to the coordinates of the center of gravity of those cells A, and are assigned to the side in which the coordinates of the center of gravity are present.

When the required graphing is completed, data processing section 253 outputs the graph data to a file (S58). As a result, time-based changes in a single cell A can be easily observed in the case of culturing cells A for a long period of time. Thus, changes in the expression levels of cells A accompanying the passage of time during culturing can be accurately and easily measured.

As has been described above, in this cell culture observation apparatus 201 and cell culture observation method, a cell culture can be observed while culturing cells A using a culture vessel 210 that is capable of maintaining cell activity. Namely, since images of cells A can be captured during culturing by the imaging step in a state in which they are housed within culture vessel 210, there is no possibility of contamination and there is no burden placed on the cells during imaging. In addition, since images are captured by dividing into measuring fields 303 corresponding to each cell A, cells can be analyzed while focusing on each measuring field 303. In addition, PC 250 recognizes the captured images according to the analysis step by reliably distinguishing each cell A according to a geometrical feature or optical feature of cells A. In other words, time-based changes that occur during the course of culturing can be accurately and continuously observed while reliably recognizing and tracking each cell A during culturing without mistaking the cells. In addition, since cells A are extracted based on a geometrical feature or optical feature, cells A to be observed can be recognized easily, thereby making it possible to shorten the time spent on observation.

In addition, a reaction of cells A to be observed can be measured on a real-time basis while changing the culturing conditions, and the presence or expressed levels of proteins or changes in the expressed levels with the passage of time can be measured accurately.

In addition, since image processing section 252 recognizes the locations of the center of gravity or surface area of cells A as geometrical features along with the luminance of cells A as an optical feature, each cell A can be clearly and precisely distinguished and recognized. Consequently, time-based changes of cells A can be reliably observed. In particular, since image processing section 252 recognizes luminance at each wavelength as an optical feature, observation of multiple types of proteins corresponding to different wavelengths can be carried out in a single observation.

Furthermore, the technical scope of the present invention is not limited by the aforementioned embodiment, and various alterations may be added within a range that does not deviate from the gist of the present invention.

For example, although the geometrical and optical features of the cells were extracted in the analysis step, at least either one may be extracted. As a result, the analysis step can be made to be compatible with the number of cells observed, such as a single cell or cell group.

In addition, although the location of the center of gravity and surface area were used as geometrical features, at least either one may be extracted. In addition, other geometrical factors allowing recognition of each cell may also be extracted without limiting to the geometrical features explained here.

Moreover, although luminance was used as an optical feature, other optical features allowing recognition of each cell may also be extracted without limiting to luminance.

In addition, although the measurement starting position and measurement ending position were determined by inputting those positions during input of the measuring range of the slide glass in the present embodiment, other methods may be used without limiting to this method. For example, a method may also be employed in which the measuring range is selected by dividing the slide glass into grids and only inputting the coordinate position where measurement is started, followed by setting the number of grids to be measured in the X and Y directions.

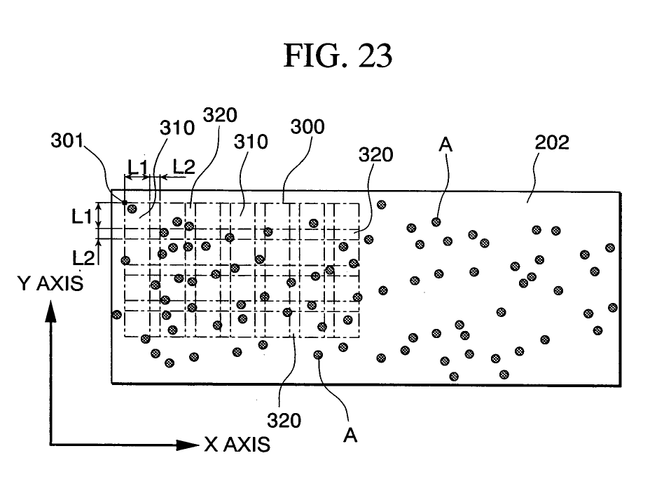

Namely, as shown in FIG. 23, squares having a side L1 as specified by an operator are defined as measured grids 310, and ranges having a length L2 as specified by an operator are defined as non-measured areas 320. The operator is then able to mutually arrange measured grids 310 and non-measured grids 320 towards the X and Y directions by specifying the starting position 301 of measured grids 310 and their number. As a result, measuring range 300 can be set. For example, the entire surface of slide glass 202 can be specified as the measuring range by specifying a value of “0” for the length L2 of non-measured area 320.

This method is preferable when using a slide glass that has been previously provided with grids.

Moreover, as an example of a different method for setting the measuring range, a method may be employed for setting the measuring range by displaying the shape of the slide glass or a pre-scanned image thereof on the monitor screen of the PC, and then specifying the measurement starting position and measurement ending position by encircling with a marker. The aforementioned pre-scanned image should be used that consists of scanning the entire surface of the slide glass with an objective lens having a low magnification, and then combining images having a short exposure time and low resolution. In addition, in the case of reading the shape of the slide glass, it is necessary to read slide glass information preliminarily stored in memory, and then specify the standards of the slide glass in order to display the shape of the slide glass on the monitor screen.

In addition, although a slide glass was used as a support, a 96-well microtiter plate or 384-well microtiter plate may also be used. At this time, cells can be cultured in each well, and fluorescent intensity from the cell culture can be measured on the lower side (bottom) of the microtiter plate.

In addition, in this case, the cells can be cultured for several days by employing a constitution consisting of, for example, lengthening the scanning stroke, providing a cover that covers the open wells of the microtiter plate, covering with a carbon dioxide supply canister and so forth.

In addition, image background can be stabilized more effectively in the present embodiment by covering the periphery with a dark curtain and so forth and setting so as to make the amount of light from the periphery of the apparatus as stable as possible.

Moreover, an erect image microscope may also be used. In this case, since it is necessary to precisely control the thickness of the culture liquid, a spacing range that does not obstruct the flow of culture liquid should be placed between the rack of the culture vessel and the glass plate to control the interval between the cells and glass plate. Furthermore, a flow straightening member may also be used instead of this spacing ring.

In the cell culture observation apparatus according to the present invention, cells can be cultured in a culture vessel capable of maintaining cell activity without removing and putting back the culture vessel from and to an incubator and so forth. In addition, the culture vessel can be moved by the movable stage. As a result, even if the imaging section is in a fixed state, images can be captured from all the locations of the culture vessel. At this time, since the imaging section captures images by dividing into each region corresponding to each cell, after the images have been captured, it is possible to focus only on a region desired to be observed such as, for example, the region of a specific cell group. Moreover, the analysis section is able to analyze the cells by extracting the cells in each region by reliably dividing into individual regions based on a geometrical feature or optical feature.

In other words, together with being able to reliably recognize and track each cell without mistaking the cells while culturing the cells in a culture vessel, the analysis section is able to track the cells by focusing on a region corresponding to each cell such as the region of a cell group. Thus, time-based changes in the resulting behavior and so forth during the culturing process can be observed accurately and continuously for each cell and each region, for example, while growing, or in other words culturing, cells for a long period of time based on the analysis results of the analysis section. In addition, since each cell or each region desired to be observed can be easily recognized, observation is easy and the time spent on observation can be reduced.

In the cell culture observation apparatus according to the present invention, the analysis section is able to recognize each cell by distinctly and precisely dividing the cells according to the location of the center of gravity or the surface area of the cells. In addition, time-based changes in the cells can be observed easily from the changes in the location of the center of gravity and surface area of the cells.

In the cell culture observation apparatus according to the present invention, together with being able to recognize cells by distinctly and precisely dividing the cells according to differences in cell luminance, the analysis section is able to easily observe time-based changes in the cells according to changes in luminance.

In the cell culture observation apparatus according to the present invention, the analysis section is able to observed multiple types of proteins and so forth corresponding to a wavelength that differs for a single observation, thereby making it possible to reduce the time and bother spent on observation and improve observation efficiency.

In the cell culture observation method according to the present invention, since images of cells during culturing can be captured in the state in which they are housed in the culture vessel in the imaging step, there is no possibility of contamination and so forth and no burden placed on the cells during imaging. In addition, since images are captured by dividing into each region corresponding to each cell, it is possible to focus on a region desired to be observed, such as only the region of a group of cells. Moreover, cells can be analyzed in the analysis step by extracting cells in each region by reliably dividing each cell based on a geometrical feature or optical feature. Thus, each cell and region can be reliably tracked while culturing the cells, and time-based changes that occur during the course of culturing can be accurately and continuously observed. In addition, in the case of having changed the culturing conditions, the reactions of the cells to be observed can also be measured on a real-time basis.

As has been explained above, according to the cell culture observation apparatus and cell culture observation method of the present invention, there is no possibility of contamination and so forth and there is no burden placed on the cells during imaging. In addition, each cell and region corresponding to each cell can be reliably recognized during culturing without error. Thus, time-based changes in the cells or region of a cell group and so forth that occur during the course of culturing can be accurately and continuously observed. Moreover, since a cell or region to be observed can be recognized easily, observation is easy and the time spent on observation can be reduced.

What id claimed is:

1. A cell culture detection apparatus comprising:
a culture vessel that houses cells together with a culture liquid,
a culturing device that cultures the cells under predetermined culturing conditions,
a detection device that detects a feature of the cells among the cells being cultured, and
a light blocking device that blocks the culture vessel from environmental light when the feature is not detected.

2. A cell culture detection apparatus according to claim 1 wherein,
the light blocking device is provided with a light blocking unit that blocks light from the periphery of the culture vessel, and
a culture vessel transport device that transports the culture vessel inside the light blocking unit.

3. A cell culture detection apparatus according to claim 1 wherein,
the light blocking device is provided with a light blocking unit that blocks light from the periphery of the culture vessel, and
a light blocking unit transport device that transports the light blocking unit to the periphery of the culture vessel.

4. A cell culture detection apparatus according to claim 1 wherein,
the culturing device is provided with a circulation pump that circulates the culture liquid,
a stirring unit that maintains the carbon dioxide in the culture liquid at a predetermined concentration by stirring the culture liquid, and
a culture liquid warming unit that controls the temperature of the culture liquid.

5. A cell culture detection apparatus according to claim 4 wherein,
the circulation pump is a non-pulsating circulation pump that pumps culture liquid without generating pressure fluctuations.

6. A cell culture detection apparatus according to claim 1 further comprising:
a measuring device that measures the level of auto-fluorescence of the culture liquid, and
a discrimination device that judges whether or not the culture liquid is degrading based on the measurement results of the measuring device.

7. A cell culture detection apparatus according to claim 6 wherein,
the discrimination device is provided with a culture liquid replacement device that replaces or replenishes the culture liquid automatically when the culture liquid has been judged have degraded.

8. A cell culture detection apparatus according to claim 6 wherein,
the discrimination device is provided with a notification device that emits an alarm when the culture liquid has been judged to have degraded.

9. A cell culture detection apparatus comprising:
a culture vessel that houses cells together with a culture liquid,
a culturing device that cultures the cells under predetermined culturing conditions, and
a detection device that detects a feature of the cells among the cells being cultured; wherein,
the culturing device has a warming device having a temperature sensor that measures the temperature of the culture vessel, and at least one of either a culture vessel warming unit that warms the culture vessel, a line warming unit that warms a line that supplies or discharges the culture liquid within the culture vessel, or a culture liquid warming unit that warms the culture liquid; and,
the warming device controls the temperature to a predetermined temperature based on the temperature measured with the temperature sensor.

10. A cell culture detection apparatus according to claim 9 further comprising:
a measuring device that measures the level of auto-fluorescence of the culture liquid, and
a discrimination device that judges whether or not the culture liquid is degrading based on the measurement results of the measuring device.

11. A cell culture detection apparatus according to claim 10 wherein,
the discrimination device is provided with a culture liquid replacement device that replaces or replenishes the culture liquid automatically when the culture liquid has been judged have degraded.

12. A cell culture detection apparatus according to claim 10 wherein,
the discrimination device is provided with a notification device that emits an alarm when the culture liquid has been judged to have degraded.

13. A cell culture detection apparatus comprising:
a culture vessel that houses cells together with a culture liquid,
a culturing device that cultures the cells under predetermined culturing conditions, and
a detection device that detects a feature of the cells among the cells being cultured; wherein,
the culturing device has a temperature sensor that measures the temperature of the culture vessel, and a culture vessel warming unit that warms the culture vessel, and
the culture vessel warming unit blows warm air towards the outer surface of the culture vessel based on the temperature measured with the temperature sensor.

14. A cell culture detection apparatus according to claim 13 further comprising:
a measuring device that measures the level of auto-fluorescence of the culture liquid, and
a discrimination device that judges whether or not the culture liquid is degrading based on the measurement results of the measuring device.

15. A cell culture detection apparatus according to claim 14 wherein,
the discrimination device is provided with a culture liquid replacement device that replaces or replenishes the culture liquid automatically when the culture liquid has been judged have degraded.

16. A cell culture detection apparatus according to claim 14 wherein,
the discrimination device is provided with a notification device that emits an alarm when the culture liquid has been judged to have degraded.

17. A cell culture observation apparatus for continuously observing time-based changes of one or a plurality or cells present on a support or in a solution; comprising:
a culture vessel that houses the cells and is capable of maintaining cell activity;
a movable stage that holds the culture vessel;
an imaging section that captures images of the cells in the culture vessel by dividing into each region corresponding to each cell; and,
an analysis section that analyzes the cells by at least extracting a geometrical feature or an optical feature of the cells within a region based on the images of each region captured by the imaging section.

18. A cell culture observation apparatus according to claim 17 wherein,
the geometrical feature is at least the location of the center of gravity or the surface area.

19. A cell culture observation apparatus according to claim 17 wherein,
the optical feature is luminance.

20. A cell culture observation apparatus according to claim 19 wherein,
the luminance is the luminance of each wavelength.

21. A cell culture observation method for continuously observing time-based changes in one or a plurality of cells present on a support or in a solution while culturing cells in a culture vessel; comprising:
an imaging step wherein images of the cells in the culture vessel are captured by dividing into each region corresponding to each cell; and
an analysis step wherein the cells are analyzed by extracting at least a geometrical feature or an optical feature of the cells in each region captured in the imaging step.
